(19) United States
(12) Patent Application Publication
(10) Pub. No.: US 2017/0202822 A1
(43) Pub. Date: Jul. 20, 2017

(54) METHODS AND COMPOSITIONS FOR ENHANCING CANCER THERAPY

(71) Applicant: THE SCRIPPS RESEARCH INSTITUTE, La Jolla, CA (US)

(72) Inventors: Antonio Fernandez Santidrian, San Diego, CA (US); Brunhilde H. Felding, San Diego, CA (US)

(21) Appl. No.: 15/326,799

(22) PCT Filed: Jul. 17, 2015
(86) PCT No.: PCT/US2015/040920

§ 371 Date: Jan. 17, 2017

Related U.S. Application Data
(60) Provisional application No. 62/025,596, filed on Jul. 17, 2014.

(51) Int. Cl. A61K 31/455; C12Q 1/48; G01N 33/574; A61K 38/45; C12N 9/10

(52) U.S. Cl. CPC A61K 31/455; A61K 38/45; C12Y 204/02012; C12N 9/1077; G01N 33/574; C12Q 1/48; A61K 48/00

(57) ABSTRACT

The present invention provides methods and compositions for enhancing efficacy of anti-hormone treatment, or for preventing cancer relapse or progression following treatment. The invention also provides methods for re-sensitizing or sensitizing treatment resistant cancer cells or patients with treatment-refractory cancer cells to continuing or starting anti-hormone treatment. Further provided in the invention are methods for prognosis or diagnosis of anti-hormone treatment effect or likelihood of cancer relapse or metastasis following anti-hormone treatment.

### CROSS-REFERENCE TO RELATED APPLICATIONS

The subject patent application claims the benefit of priority to U.S. Provisional Patent Application No. 62/025,596 (filed Jul. 17, 2014). The full disclosure of the priority application is incorporated herein by reference in its entirety and for all purposes.

### STATEMENT OF GOVERNMENT SUPPORT

This invention was made with U.S. government support under Grant Nos. R01CA170737 and R01CA170140 awarded by the National Institutes of Health. The U.S. government has certain rights in the invention.

### BACKGROUND OF THE INVENTION

Cancer is one of the leading causes of death, and metastatic cancer is often incurable. For example, breast cancer metastasis to lungs, liver, bone and brain is the primary cause of death in breast cancer patients. It involves cancer cell dissemination via the blood stream and lymphatic system, and depends on adhesive and invasive tumor cell functions and their ability to survive and proliferate at target sites. The mortality in breast cancer remains high, despite advances in diagnosis and treatment. A major underlying problem is that breast cancer frequently recurs, often years after apparently successful therapy. About 90% of deaths are caused by metastasis for which no effective therapies exist. For example, triple negative breast cancer is the most aggressive breast cancer defined by the lack of expression of estrogen receptor alpha (ER alpha), progesterone receptor (PR) and receptor tyrosine-protein kinase erbB-2 (HER2). Patients with triple negative breast cancer are not treated with anti-hormone therapy such as Tamoxifen or aromatase inhibitors, because their tumors lack ER alpha expression. At present, no available treatment can effectively cure triple negative breast cancer.

Despite improvements in breast cancer surgery and treatment, the mortality of breast cancer patients has largely remained unchanged. A major underlying problem is that some tumor cells have and many develop treatment resistance, leading to disease recurrence, often years after initial adjuvant therapy was apparently successful. This includes anti-hormone treatment, the major targeted therapy and standard of care for hormone receptor positive breast cancers, which comprise the majority of breast cancers overall.

There is a need in the art for means that can more effectively treat cancer by preventing tumor metastases and treatment resistance. The instant invention is directed to addressing this and other needs.

### SUMMARY OF THE INVENTION

In one aspect, the invention provides methods for re-sensitizing or sensitizing a population of treatment resistant cancer cells to an anti-hormone therapy. The methods involve contacting the treatment resistant cancer cells with a compound that upregulates NAD⁺ or NAD⁺/NADH redox balance in the cells, thereby re-sensitizing or sensitizing the cancer cells. Some of the methods are directed to treatment of cancer cells that are estrogen receptor (ER) positive, e.g., ER-positive breast cancer or ovarian cancer cells. Some other methods are directed to treatment of cancer cells that are estrogen receptor (ER) negative, e.g., ER-negative breast cancer or ovarian cancer cells.

In some methods of the invention, the treatment resistant cancer cells are present in a patient. For example, the treatment can be directed to cancer cells present in a patient who has undergone treatment with an anti-hormone therapy. In some methods, the anti-hormone therapy is treatment with Tamoxifen or another compound capable of reducing (or compounds aimed to reduce) estrogen levels estrogen levels systemically. In some methods, NAD⁺ or NAD⁺/NADH redox balance is upregulated via enhanced NAD⁺ salvage pathway synthesis, enhanced NAD⁺ de novo synthesis, enhanced NAMPT activation, or enhanced NAMPT cellular level. In some of these methods, the enhanced NAD⁺ salvage pathway synthesis is via administration of a NAD precursor. The NAD precursor employed in these methods can be, e.g., nicotinamide (NAM), nicotinic acid (Na), or nicotinamide riboside (NR).

In some other methods of the invention, NAD⁺ or NAD⁺/NADH redox balance is upregulated by introducing into the cancer cells an agent that upregulates NAMPT cellular level. The agent suitable for these methods can be, e.g., a polynucleotide or expression vector encoding NAMPT. In some of these methods, the polynucleotide can be administered to the patient via tumor marker targeted gene delivery. In some other methods, the polynucleotide is administered to the patient via stem cell-based gene delivery. In some other methods, upregulated NAMPT cellular level is achieved by inducing glucose deprivation in blood or inhibiting consumption of glucose by cancer cells.

In another aspect, the invention provides methods for enhancing anti-hormone therapy efficacy or preventing cancer relapse or progression in a cancer patient. These methods entail administering to a patient undergoing treatment with, having been treated, or never treated with anti-hormone therapy an agent which upregulates NAD⁺ or NAD⁺/NADH redox balance, thereby enhancing anti-hormone therapy efficacy or preventing cancer relapse or progression in the patient. In some of these methods, the cancer is an estrogen receptor (ER) positive breast cancer or ovarian cancer. In some other methods, the cancer is an estrogen receptor (ER) negative breast cancer or ovarian cancer. Some of the methods are directed to treating patient who have invasive or non-invasive primary tumor, have or will have surgical removal of a primary tumor, or have metastatic cancer. Some of the methods are specifically directed to patients who have undergone anti-hormone therapy. Some methods are specifically directed to patients who are concurrently undergoing anti-hormone therapy. Some other methods are specifically directed to patients who have never undergone anti-hormone therapy. In various methods, the patient can be administered the agent prior to, simultaneously with, or subsequent to the anti-hormone therapy.

In some methods, upregulation of NAD⁺ or NAD⁺/NADH redox balance is via modulation of a NAD⁺ redox pathway or modulation of a NAD⁺ non-redox pathway. In some of these methods, the NAD⁺ or NAD⁺/NADH redox pathway is glycolysis pathway, pentose phosphate pathway, a cytosolic NAD regeneration pathway, citric acid cycle pathway, glutaminolysis pathway, beta-oxidation pathway, mitochondrial respiration pathway, a lipid synthesis pathway, nicotinamide nucleotide transhydrogenase pathway, or a pathway involving a NADH dehydrogenase pathway. In some methods, the NAD⁺ non-redox pathway is a NAD⁺ synthesis pathway, a NAD⁺ consumption pathway or a NAD⁺/NADH dependent pathway. In some of these methods, the NAD⁺ synthesis pathway, the NAD⁺ consumption pathway, or the NAD+/NADH dependent pathway is modulated via a NAD⁺ precursor, an enzyme involved in NAD⁺ synthesis or an enzyme involved in NAD⁺ consumption. In these methods, the NAD⁺ precursor can be nicotinamide (NAM), nicotinic acid (Na), nicotinamide-riboside (NR) or tryptophan. In some of these methods, the NAD⁺ precursor is an intermediate metabolite in the pathway of NAD⁺ synthesis. In some methods, the enzyme involved in NAD⁺ synthesis is NAMPT. In some of the methods, the enzyme involved in NAD⁺ consumption is PARP, Sirtuins or CD38.

In another aspect, the invention provides methods for treating a cancer in a patient. The methods involve (1) treating the patient with an anti-hormone therapy, and (2) administering to the subject a compound which upregulates NAD⁺ or NAD⁺/NADH redox balance. In some of these methods, the cancer to be treated is an estrogen receptor (ER) positive cancer, e.g., ER-positive breast cancer or ovarian cancer. In some other methods, the cancer to be treated is an estrogen receptor (ER) negative cancer, e.g., ER-negative breast cancer or ovarian cancer. In some methods, the employed anti-hormone therapy entails administration of a pharmaceutical composition comprising a therapeutically effective amount of an antagonist compound of the estrogen receptor. In some methods, the employed anti-hormone therapy is treatment with Tamoxifen or another compound capable of reducing estrogen levels systemically. In various embodiments, the compound upregulating NAD⁺ or NAD⁺/NADH redox is administered to the subject prior to, concurrently with, or subsequent to treatment with the anti-hormone therapy. In some methods, the patient is first treated with the anti-hormone therapy prior to administering the compound which upregulates NAD⁺ or NAD⁺/NADH redox balance. Some of these methods can additionally include examining the patient for resistance to the anti-hormone treatment after step (1). Some of the methods are directed to patients who have developed resistance to anti-hormone treatment prior to administering the compound. Some methods of the invention can further include continuing treating the patient with an anti-hormone therapy after step (2).

In some of the cancer-treating methods of the invention, upregulation of NAD⁺ or NAD⁺/NADH redox balance is via modulation of a NAD⁺ redox pathway or modulation of a NAD⁺ non-redox pathway. For example, the NAD⁺/NADH redox pathway to be modulated can be glycolysis pathway, pentose phosphate pathway, a cytosolic NAD⁺ regeneration pathway, citric acid cycle pathway, glutaminolysis pathway, Beta-oxidation pathway, mitochondrial respiration pathway, a lipid synthesis pathway, nicotinamide nucleotide transhydrogenase pathway, or a pathway involving a NADH dehydrogenase pathway. In some other methods, the NAD⁺ non-redox pathway to be modulated can be, e.g., NAD⁺ synthesis pathway, a NAD⁺ consumption pathway, or a NAD⁺/NADH dependent pathway. In some of these methods, the NAD⁺ synthesis pathway, the NAD⁺ consumption pathway, or the NAD⁺/NADH dependent pathway can be modulated via a NAD⁺ precursor, an enzyme involved in NAD⁺ synthesis, or an enzyme involved in NAD⁺ consumption. In some embodiments, the employed NAD⁺ precursor can be, e.g., nicotinamide (NAM), nicotinic acid (Na), nicotinamide riboside (NR) or tryptophan. In some other embodiments, the employed NAD⁺ precursor is an intermediate metabolite in NAD⁺ synthesis pathway. In some methods, the enzyme involved in NAD synthesis is NAMPT. In some other methods, the enzyme involved in NAD⁺ consumption is PARP, Sirtuins or CD38.

In still another aspect, the invention provides methods for prognosing or diagnosing cancer relapse or distant metastasis after anti-hormone therapy in a cancer patient. The methods entail (a) determining NAMPT level, NAD⁺ level, ratio of NAD⁺/NADH levels in the cancer of the patient, or the level or activity of an enzyme involved in NAD⁺ consumption, and (b) correlating the determined NAMPT level, NAD⁺ level, ratio of NAD⁺/NADH levels, or the level or activity of the enzyme involved in NAD⁺ consumption, with an increased risk of cancer relapse or distant metastasis, or lack thereof, in the patient. In a related aspect, the invention provides methods for prognosing or diagnosing effect of anti-hormone therapy in a cancer patient. These methods involve (a) determining NAMPT level, NAD level, ratio of NAD⁺/NADH levels, or the level or activity of an enzyme involved in NAD⁺ consumption, in the cancer of the patient, and (b) prognosing or diagnosing from the determined NAMPT level, ratio of NAD⁺/NADH levels, or the level or activity of the enzyme involved in NAD⁺ consumption, a post-treatment effect of anti-hormone therapy in the patient. In these methods, the enzyme involved in NAD⁺ consumption can be, e.g., PARP, Sirtuins or CD38. Some of these methods are directed to prognosis or diagnosis of breast cancer or ovarian cancer. In some of these methods, the cancer is ER-positive breast cancer or low grade breast cancer.

In some methods, NAMPT level, NAD⁺ level or ratio of NAD⁺/NADH levels is determined prior to or during the anti-hormone therapy. In some methods, step (b) comprises comparing the determined NAMPT level, NAD⁺ level or ratio of NAD⁺/NADH levels in the cancer of the patient to one or more reference levels associated with cancer relapse or distant metastasis. In some methods, step (b) further comprises assigning the determined level in the cancer of the subject a value or designation providing an indication whether the patient has an increased risk of cancer relapse or distant metastasis. In some of these methods, the assigned value or designation is based on a normalized scale of values associated with a range of levels in cancer patients treated by anti-hormone therapy who have an increased risk of cancer relapse or distant metastasis.

A further understanding of the nature and advantages of the present invention may be realized by reference to the remaining portions of the specification and claims.

### DESCRIPTION OF THE DRAWINGS

FIG. 1 outlines the nicotinamide adenine dinucleotide (NAD⁺) synthesis and salvage pathway. Vitamin B3: Nicotinamide (NAM, also known as niacinamide) is a NAD⁺ precursor obtained from the diet. NAM is also a product of NAD⁺ consumption. Nicotinamide phosphoribosyltransferase (NAMPT) is an enzyme essential for the utilization and recycling of NAM. NAMPT catalyzes the condensation of NAM and phosphoribosyl pyrophosphate (PRPP) to yield nicotinamide mononucleotide (NMN⁺), the first step in the biosynthesis of NAD⁺. Nicotinamide mononucleotide adenylyltransferase (NMNAT) catalyzes the second and last step of NAD⁺ synthesis.

FIG. 2 shows NAMPT expression levels correlate with Estrogen Receptor alpha status in human breast tumors. Box plot analysis of NAMPT mRNA expression in Estrogen Receptor (ER) negative (n=395) and ER positive tumors (n=1225) (P<0.00001).

FIG. 3 shows that low NAMPT expression induces 4-hydroxytamoxifen resistance in MCF7 and T47D, human ER-positive breast cancer cell lines. NAMPT knockdown (shNAMPT) reduced NAMPT (A) mRNA and (B) protein expression in MCF7 and T47D cells compared to controls transduced with scrambled shRNA (shCtrl). (A) NAMPT mRNA levels were analyzed by real time PCR and are expressed relative to beta-D-glucuronidase (GUSB) (***P<0.001) (n=3). (B) NAMPT protein expression was analyzed by Western blot analysis. Quantification of NAMPT protein was related to β-tubulin expression. (C) NAMPT knockdown (shNAMPT) reduced cellular NAD⁺ in MCF7 and T47D cells compared to controls transduced with scrambled shRNA (shCtrl). Cellular NAD was analyzed in whole cell extracts of 1×10⁶ cells. Metabolite concentrations were determined using a NAD+/NADH fluorescence detection kit (Cell Technology, Inc) and normalized to protein content. (D) Proliferation of control (shCtrl) vs NAMPT-knockdown (shNAMPT) MCF7 and T47D cells, untreated or treated with 0.001, 0.05, 0.1, 1, or 5 μM 4-hydroxytamoxifen (tamoxifen active metabolite) for 14 days. Proliferation was measured based on crystal violet staining and is expressed as % of proliferation of untreated cells. Groups were compared by unpaired two-tailed Student's t-test in n=4 (***P<0.001, **P<0.01 *P<0.05).

FIG. 4 shows that treatment with nicotinamide, a NAD⁺ precursor, blocks low NAMPT-induced tamoxifen resistance in MCF7 and T47D cells, and that NAD⁺ precursor treatment or NAMPT downregulation do not affect estrogen receptor alpha (ERα) expression and nuclear localization in MCF7 cells. (A) Effect of nicotinamide treatment (10 mM NAM) on proliferation of control (shCtrl) vs NAMPT-knockdown (shNAMPT) MCF7 and T47D cells exposed to 1 μM or 0.1 μM 4-hydroxytamoxifen (tamoxifen active metabolite) respectively for 14 days. Proliferation was measured based on crystal violet staining and is expressed as % of proliferation of untreated cells (no 4-hydroxytamoxifen, no NAM). Groups were compared by unpaired two-tailed Student's t-test in n=4 (***P<0.001, **P<0.01 *P<0.05). (B) NAMPT KD cells present reduced absolute levels of NAD′ and nicotinamide treatment induces NAD⁺ and NADH levels in both control (CT) or NAMPT KD (shNAMPT) breast cancer cells. NAD⁺ and NADH were analyzed independently in whole cell extracts of 1×10⁶ cells. Metabolite concentrations were determined using a NAD⁺/NADH fluorescence detection kit (Cell Technology, Inc). (C) Distribution of ERα in MCF7 shCT or shNAMPT cells, measured after 7 days of cell treatment with 10 mM nicotinamide in EMEM medium, supplemented with 10% FBS. ERα localization was detected by immunofluorescence using anti-ERα clone SP1 (Thermo Fisher). Nuclei were detected by DAPI staining. Representative images are shown.

FIG. 5 shows that NAD⁺ precursors nicotinamide and nicotinamide riboside restore tamoxifen sensitivity in ER+/NAMPT-low breast cancer cells. Nicotinamide riboside (NR) was more efficient than nicotinamide in blocking low-NAMPT-induced tamoxifen resistance. Proliferation of control (shCT) vs NAMPT-knockdown (shNAMPT) MCF7 cells treated with 1 or 5 μM 4-hydroxytamoxifen (tamoxifen active metabolite) for 7 days with or without (A) 1, 5 or 10 mM nicotinamide (NAM) or (B) 1 or 5 mM nicotinamide riboside (NR). Proliferation was measured based on crystal violet staining and is expressed as % of proliferation of untreated cells (no 4-hydroxytamoxifen). Groups were compared by unpaired two-tailed Student's t-test in n=4 (***P<0.001, **P<0.01 *P<0.05).

FIG. 6 shows that low NAMPT expression induces estrogen-independent growth in MCF7 ER-positive human breast cancer cells. (A) Growth of control (shCtrl) vs NAMPT knockdown (shNAMPT) MCF7 cells cultured for 7 days in EMEM medium supplemented with 10% FBS, or in phenol red-free EMEM supplemented with 10% charcoal-stripped estrogen-free FBS. Growth was measured by crystal violet staining after 7 days in culture. Groups were compared by unpaired two-tailed Student's t-test in n=3 (***P<0.001). (B) Growth of control (shCtrl) vs NAMPT knockdown (shNAMPT) MCF7 cells cultured in estrogen-free, phenol red-freeEMEM medium supplemented with 10% charcoal-stripped FBS. MCF7 cells were not treated or treated with 10 nM 17-β-estradiol (E2) in the absence or presence of 1 μM 4-hydroxytamoxifen (E2, E2+4-OHT) for 7 days, with our without 10 mM nicotinamide (NAM). Growth was measured by crystal violet staining after 7 days in culture. Groups were compared by unpaired two-tailed Student's t-test in n=3 (*P<0.05,***P<0.01,***P<0.001). (C) Changes in NAD⁺ levels impact the subcellular localization of ERα. Distribution of ERα in MCF-7 shCT and MCF-7 shNAMPT cells, starved for estrogens for 72 h prior to treatment with 10 nM 17-β-estradiol (E2) or 1 nM E2 plus 10 mM NAM for 24 h. Estrogen starvation and treatment were performed in phenol red-free EMEM supplemented with 10% charcoal-stripped FBS. ERα was detected by immunofluorescence using Pierce anti-ERα antibody (MA1-39539). Representative images are shown for all conditions.

FIG. 7 shows that low NAMPT expression in MCF7 ER-positive human breast cancer cells induces estrogen-independent tumorigenicity in the mouse model. Size of mammary fat pad tumors induced by implantation of MCF7 control (shCT) or NAMPT knockdown (shNAMPT) cells in SCID mice. Mice were not implanted with 17-β-estradiol pellets to eliminate estrogen growth stimulation, necessary for tumor formation by MCF7 control cells. Tumor size was analyzed by caliper measurements (mm³). In box plots, top line denotes the 75% quartile, bottom line the 25% quartile, middle line the median, and whiskers the minima and maxima. Group comparisons by nonparametric Mann-Whitney test (*** P<0.001) (n=7).

FIG. 8 shows that treatment with nicotinamide, a NAD+ precursor, blocks resistance of MDA-MB-231 cells (triple negative human breast cancer cell line) to tamoxifen. Proliferation of triple negative breast MDA-MB-231 cells, untreated or treated with 0.5, 1 or 5 μM 4-hydroxytamoxifen (tamoxifen active metabolite) for 14 days, with or without treatment with 10 mM nicotinamide (NAM). Proliferation was measured based on crystal violet staining and is expressed as % of proliferation of untreated cells (no 4-hydroxytamoxifen, no NAM). Groups were compared by unpaired two-tailed Student's t-test in n=3 (**P<0.01).

FIG. 9 shows that glucose deprivation upregulates NAMPT expression in human breast cancer cells. NAMPT mRNA expression levels in parental MCF7 cells cultured in media containing 5 mM or 0.1 mM glucose and 10% dialyzed FBS for 48 hours. NAMPT mRNA levels were analyzed by real time PCR and are expressed relative to GUSB. Groups were compared by unpaired two-tailed Student's t-test in n=3 (***P<0.001).

FIG. 10 shows that high NAMPT levels correlate with good prognosis in low grade and in ER-positive breast cancers. Kaplan Meier analysis over 10 years of recurrence free survival (RFS) (left panel) or distant metastasis free survival (DMFS) (right panel) in patients with (A) Estrogen Receptor (ER)-positive tumors, or (B) grade 1 breast cancer (independently of receptor status), expressing either high NAMPT (top line, with a Log 2 relative expression between −0.15 and 3.97) or low NAMPT (bottom line, with a Log 2 relative expression between −2.35 and −0.15); (A) RFS in high-NAMPT tumors (n=52), or low-NAMPT tumors (n=138), (p=0.00091); DMFS in high-NAMPT tumors (n=66), or low-NAMPT tumors (n=75), (p=0.00392); (B) RFS in high-NAMPT tumors (n=200), or low-NAMPT tumors (n=538), (p=0.00014); DMFS in high-NAMPT tumors (n=361), or low-NAMPT tumors (n=495), (p=0.00005).

FIG. 11 shows that high NAMPT levels correlate with good prognosis in tamoxifen treated patients with ER-positive breast cancer. Kaplan Meier analysis over 10 years of distant metastasis free survival (DMFS) in patients with untreated or tamoxifen treated ER-positive breast cancer expressing high NAMPT (top line, with a Log 2 relative expression between −0.355 and 3.858) or low NAMPT (bottom line, with a Log 2 relative expression between −2.351 and −0.355). DMFS in untreated high-NAMPT tumors (n=197), vs low-NAMPT tumors (n=240), (P=0.08609). DMFS in tamoxifen treated high-NAMPT tumors (n=196), vs low-NAMPT tumors (n=103), (P=0.08609).

### DETAILED DESCRIPTION OF THE INVENTION

### I. Introduction

Adjuvant anti-hormone therapy after breast cancer surgery increases life expectancy. Treatment with estrogen receptor (ER) antagonists such as tamoxifen can reduce the risk of developing local and metastatic recurrence in pre-menopausal patients with ER-positive (ER-alpha) breast cancer. However, an important portion of ER-positive patients (30 to 40%) will develop distant metastasis, despite anti-hormone therapy. Aromatase inhibitors, compounds aimed at reducing estrogen levels systemically, have been shown to be a more effective treatment than tamoxifen for ER-positive breast cancer in post-menopausal patients. Nevertheless, anti-hormone adjuvant therapy is not recommended to be taken for longer than 5 years. Thus, for all of these reasons, there is a clinical need for improving current therapies, and for identifying patients who had ER-positive tumors and who are likely to relapse after their anti-hormone treatment ends.

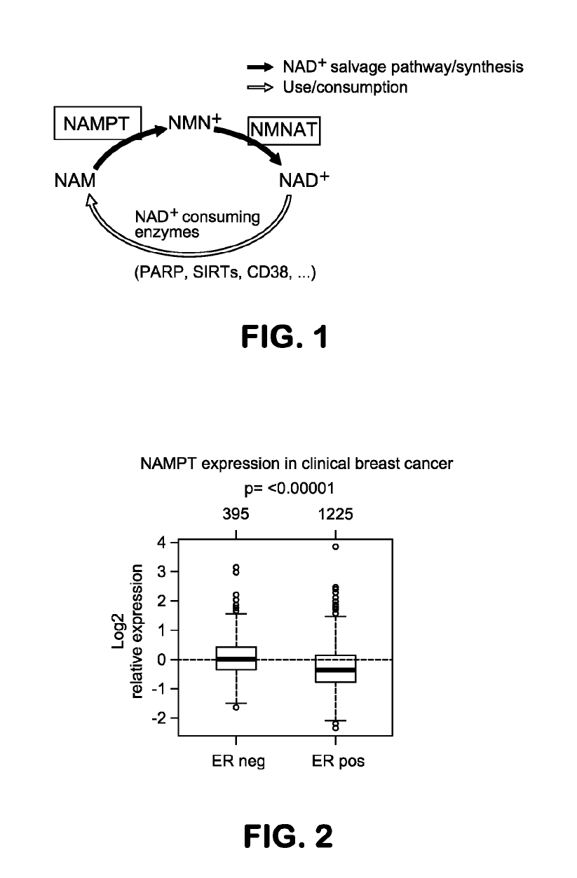

Nicotinamide-phosphoribosyl transferase (NAMPT), also known as pre-B-cell colony-enhancing factor 1 (PBEF1) or visfatin, is a key enzyme in nicotinamide adenine dinucleotide (NAD⁺) production from dietary NAD⁺ precursors, as well as in NAD⁺ recovery via the NAD⁺ salvage pathway. NAMPT catalyzes the conversion of nicotinamide (NAM), also known as niacinamide or vitamin B3, to nicotinamide mononucleotide (NMN⁺) using phospho ribosyl pyrophosphate (PRPP) as a co-substrate. NMN⁺ is then converted to NAD⁺ by nicotinamide nucleotide adenylyltransferases (NMNAT). In addition to hundreds of metabolic reactions, NAD⁺ is also used by NAD⁺ consuming enzymes, such as poly (ADP-ribose) polymerases (PARPs), Sirtuins and CD38 (FIG. 1). These proteins are involved in DNA damage repair mechanisms, cellular proliferation, autophagy, apoptosis, cellular metabolism, and various other pathways. NAD⁺ consuming enzymes produce NAM as a byproduct of the reaction. NAMPT is an essential protein in the recovery of cellular NAD⁺ levels. NAD⁺ can be reduced to NADH through catabolic reactions, mainly in glycolysis, glutaminolysis and the TCA cycle. NADH is used as a cofactor of enzymatic reactions or by mitochondrial complex I in the electron transfer chain for energy production.

Tumor cells, specifically highly proliferative ER-negative or basal-like breast cancer cells, generally accumulate high levels of DNA damage, genomic instability, and have increased dependence of PARP activity. PARPs are NAD⁺ consuming DNA damage repair proteins that correlate with the high needs of the tumor cells for NAD⁺ to maintain cell viability. It has been suggested in the art that high NAMPT expression will enhance tumor cell survival, even under stress, by supporting cellular NAD⁺ levels. See, e.g., Krishnakumar et al., Mol. Cell 39, 8-24 (2010); Bajrami et al., EMBO Mol. Med. 4, 1087-96 (2012); and Hsu et al., Autophagy 5, 1229-1231 (2009). Consistently, the inventors analyzed breast cancer gene array databases in combination with outcome data from 1881 breast cancer patients reported by Ringnér et al. (PLoS One 6, e17911, 2011) and found that ER-negative breast cancers have significantly higher levels of NAMPT expression than ER-positive breast cancers (FIG. 2), in line with other reports in the art (e.g., Lee et al., Cancer Epidemiol. Biomarkers Prev. 20, 1892-901, 2011). It has also been suggested that high NAMPT levels, which induce an increased NAD⁺ level or faster recovery of NAD⁺, can induce resistance to genotoxic-therapy, the basis for many chemotherapeutic approaches. See, e.g., Folgueira et al., Clin. Cancer Res. 11, 7434-43 (2005). Thus, chemical inhibition of NAMPT alone, aimed to induce a dramatic cellular depletion of NAD⁺, or in combination with PARP inhibitors has been proposed as a therapeutic approach for triple negative breast cancer (See, e.g., Bajrami et al., EMBO Mol. Med. 4, 1087-96, 2012).

Mitochondrial NADH dehydrogenase (Complex I) is the initial enzyme in the mitochondrial electron transport chain (ETC). Using NADH as a substrate, complex I transfers an electron to ubiquinone, pumping a proton into the mitochondrial intramembrane space which ultimately leads to ATP production by ATP-synthase. Complex I also regulates the mitochondrial and cellular NAD⁺/NADH balance through its main activity as NADH dehydrogenase. Enhancement of mitochondrial complex I activity that leads to increased cellular NAD⁺ levels inhibits the aggressive phenotype in breast cancer cells (Santidrian et al., J. Clin. Invest. 123: 1068-1081, 2013). However it is known in the art that enhancement of mitochondrial complex I activity, through increase of NAD⁺ levels, also blocks the anti-proliferative effect of approaches that induce metabolic stress (Santidrian et al., J. Clin. Invest. 123: 1068-1081, 2013), and dramatically inhibits the therapeutic effect of anticancer drugs known as biguanides (e.g., Metformin) (Birsoy et al., Nature 508: 108-112, 2014).

In view of the above teachings in the art, one would expect that low expression of NAMPT in ER-positive breast cancer cells could be associated with low NAD⁺ levels or a low capacity to recover NAD⁺. It would also be expected that low expression of NAMPT could set the cells up for good responsiveness to genotoxic and cell stress-inducing therapeutic treatments, including the most widely used ER-targeted anti-hormone therapies or approaches. One would further expect that treatment with NAD⁺ precursors would inhibit efficacy of anti-hormone therapy in ER-positive breast cancers and counteract growth-blocking effects of this therapy.

The present invention is predicated in part on the inventors' surprising discovery that the efficacy of anti-hormone therapy can be significantly and substantially enhanced by upregulating NAD⁺ levels. As noted above, it was believed in the art prior to the present invention that inhibition of NAD synthesis and salvage pathways is a promising anti-cancer therapy. See, e.g., Galli et al., J. Med. Chem. 56:6279-6296, 2013; and Shackelford et al., Genes & Cancer 4: 447-456, 2013. While NAD⁺ precursor treatment, as a single agent, was reported to be able to inhibit tumor progression through modulation of mTOR activity and induction of autophagy (Santidrian et al., J. Clin. Invest. 123: 1068-1081, 2013), it was also known in the art that autophagy induction can promote tumorigenesis by supporting tumor cell survival under stress. See, e.g., White, Nat. Rev. Cancer 12, 401-10, 2012. Such stress can be induced by cancer therapies. Specifically, it has been shown that autophagy induction may inhibit the effects of anti-hormone treatment, which is the standard of care for ER-positive breast cancers (see, e.g., Cook et al., Expert Rev. Anticancer Ther. 11, 1283-94, 2011). Thus, what was known in the art would suggest that NAD⁺ precursor treatment could interfere with anti-hormone treatment efficacy, and that this treatment should therefore not be combined with other cancer therapies such as anti-hormone treatment.

Contrary to what would be expected by the artisans, the present inventors demonstrated that NAD⁺ precursor treatment significantly and substantially enhances efficacy of anti-hormone therapy. It was found that NAD⁺ precursor treatment can actually sensitize in otherwise insensitive breast cancer cells (e.g., triple negative breast cancer or non-responsive ER+ breast cancer cells) to anti-hormone therapy, as well as increase sensitivity in ER+ breast cancer cells, and re-sensitize breast cancer cells (e.g. ER-positive breast cancer cells) that have become refractory to anti-hormone therapy. As detailed herein, it was also found that expression levels of nicotinamide-phosphoribosyl transferase (NAMPT), a key enzyme in the NAD⁺ synthesis and salvage pathway, positively correlate with the efficacy of anti-hormone therapy in breast cancer. The inventors further found that enhancement of NAD⁺ levels, NAD⁺ synthesis or salvage pathway activity, or NAMPT activation can drastically reduce treatment resistance and cancer recurrence in patients with ER-positive breast cancer, who have been treated with anti-hormone therapy. Moreover, the inventors found that triple negative breast cancer cells treated with NAD⁺ precursors became responsive to anti-hormone therapy. In summary, the inventors' work demonstrated that NAD⁺ precursor treatment can re-sensitize or sensitize tumor cells to anti-hormone therapy that are or have become treatment refractory, and that NAD⁺ precursor treatment could benefit patients harboring tumor cells that are or have become resistant to anti-hormone treatment.

In accordance with these discoveries, the present invention provides methods for enhancing efficacy of anti-hormone treatment of ER-positive cancers, or re-sensitizing or sensitizing resistant cancer cells to anti-hormone therapy. The methods entail administering to patients who have undergone or are currently receiving anti-hormone treatment a compound which can up-regulate the NAD⁺/NADH redox ratio. As detailed herein, the upregulation of NAD⁺/NADH balance can be achieved via, e.g., upregulating NAD⁺ levels, enhancing NAD⁺ synthesis or salvage pathways, or activating NAMPT or inducing NAMPT expression. Additionally, the data disclosed herein indicate that NAMPT expression in tumors could be used as a biomarker to determine the probability of cancer progression during and cancer recurrence after anti-hormone therapy, e.g., Tamoxifen treatment. The invention also provides diagnostic tools for assessing the likelihood of recurrence of cancer in patients treated with anti-hormone therapy.

The combination of NAD⁺ upregulation with anti-hormone treatment overcomes critical hurdles in the standard of care therapy for patients with ER-positive breast cancer, the majority of all breast cancer cases. As demonstrated herein, it also represents a new treatment option for ER-negative breast cancer, one of the most aggressive subtypes of breast cancer. These critical hurdles that currently limit patient survival, and which can be overcome by methods of the invention, include disease progression, disease recurrence, treatment resistance, and cessation of initial treatment responsiveness. They also include the need for identification of patients based on molecular features who have a high risk of disease progression or recurrence at the beginning of and throughout treatment. They additionally include identification of molecular markers as early, as well as continuous indicators of treatment responsiveness.

A combination of NAD⁺ precursor treatment and anti-hormone therapy can be most beneficial for treatment of estrogen responsive cancers to enhance patient outcomes. Considering the non-toxic nature of NAD⁺ precursor treatment, combining NAD⁺ precursors with anti-hormone therapy, and extending NAD⁺ precursor treatment beyond the duration of the anti-hormone therapy, this treatment combination can significantly enhance survival in breast cancer patients and patients with other hormone responsive tumors. Such a treatment regimen is suitable for patients with ER-positive tumors that express high levels as well as those that express low levels of NAMPT. For patients with low NAMPT expressing tumors, which will have poorer prognosis, NAD⁺ precursor treatment can significantly extend survival. Additionally, since NAD⁺ precursor treatment can enhance the anti-proliferative effects of anti-hormone therapy, it will also enable clinical efficiency of lower anti-hormone therapy doses and allow for extended use of anti-hormone therapy beyond the 5-year mark that is presently well established in the art. The extension of the treatment period by the combination therapy discovered by the inventors can optimize overall outcome while preserving quality of life. Finally, the combination of NAD⁺ precursor treatment and anti-hormone therapy can be beneficial for ER-negative cancer patients which are usually not treated with anti-hormone therapy prior to the present invention.

The following sections provide more detailed guidance for practicing the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

Autophagy (or autophagocytosis) is the basic catabolic mechanism that involves cell degradation of unnecessary or dysfunctional cellular components through the actions of lysosomes. The breakdown of cellular components can ensure cellular survival during starvation by maintaining cellular energy levels. Autophagy, if regulated, ensures the synthesis, degradation and recycling of cellular components. During this process, targeted cytoplasmic constituents are isolated from the rest of the cell within the autophagosome, which are then fused with lysosomes and degraded or recycled. There are three different forms of autophagy that are commonly described; macroautophagy, microautophagy and chaperone-mediated autophagy. In the context of disease, autophagy has been seen as an adaptive response to survival, whereas in other cases it appears to promote cell death and morbidity.

Unless otherwise noted, the terms “patient”, “subject” and “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

“Treating” or “treatment” includes the administration of the antibody compositions, compounds or agents of the present invention to prevent or delay the onset of the symptoms, complications, or biochemical indicia of a disease, alleviating the symptoms or arresting or inhibiting further development of the disease, condition, or disorder (e.g., cancer, metastatic cancer, or metastatic breast cancer). Treatment can be prophylactic (to prevent or delay the onset of the disease, or to prevent the manifestation of clinical or subclinical symptoms thereof) or therapeutic suppression or alleviation of symptoms after the manifestation of the disease.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancerous” or “malignant cell” is understood as a cell having specific structural properties, lacking differentiation and being capable of invasion and metastasis. Examples of cancers are breast, lung, brain, bone, liver, kidney, colon, prostate, ovarian, and pancreatic cancer and melanoma. See, e.g., DeVita et al., Eds., Cancer Principles and Practice of Oncology, 6th. Ed., Lippincott Williams & Wilkins, Philadelphia, Pa., 2001.

“Advanced cancer” means cancer that is no longer localized to the primary tumor site, or a cancer that is Stage III or IV according to the American Joint Committee on Cancer (AJCC).

“Metastasis” or “metastatic” refers to the ability of tumor cells to spread from a primary tumor (e.g., a breast cancer) to establish secondary tumor lesions in locations that are distant from the site where the primary tumor occurs or is established (e.g., lung, liver, bone or brain). A “metastatic” cell typically can invade and destroy the neighboring tissue or body structures around the primary tumor site.

NAD⁺ synthesis, or de novo production, is one of the two metabolic pathways by which NAD⁺ is synthesized. Most organisms synthesize NAD⁺ from simple components. The specific set of reactions differs among organisms, but a common feature is the generation of quinolinic acid (QA) from an amino acid, either tryptophan (Trp) in animals and some bacteria, or aspartic acid in some bacteria and plants. The quinolinic acid is converted to nicotinic acid mononucleotide (NaMN) by transfer of a phosphoribose moiety. An adenylate moiety is then transferred to form nicotinic acid adenine dinucleotide (NaAD). Finally, the nicotinic acid moiety in NaAD is amidated to a nicotinamide (NAM) moiety, forming nicotinamide adenine dinucleotide. In a further step, some NAD′ is converted into NADP⁺ by NAD⁺ kinase, which phosphorylates NAD⁺. In most organisms, this enzyme uses ATP as the source of the phosphate group, although several bacteria (such as Mycobacterium tuberculosis) and a hyperthermophilic archaeon Pyrococcus horikoshii use inorganic polyphosphate as an alternative phosphoryl donor.

NAD⁺ salvage pathways refer to the processes which recycle preformed components such as nicotinamide back to NAD⁺. Besides assembling NAD⁺ de novo from simple amino acid precursors, cells also salvage preformed compounds containing nicotinamide. Although other precursors are known, the three natural compounds containing the nicotinamide ring and used in these salvage metabolic pathways are nicotinic acid (Na), nicotinamide (NAM) and nicotinamide riboside (NR). These compounds can be taken up from the diet, where the mixture of nicotinic acid and nicotinamide are called vitamin B3 or niacin. However, these compounds are also produced within cells, when the nicotinamide moiety is released from NAD⁺ in ADP-ribose transfer reactions. Indeed, the enzymes involved in these salvage pathways appear to be concentrated in the cell nucleus, which may compensate for the high level of reactions that consume NAD⁺ in this organelle. Cells can also take up extracellular NAD⁺ from their surroundings.

The term “treating” or “alleviating” includes the administration of compounds or agents to a subject to prevent or delay the onset of the symptoms, complications, or biochemical indicia of a disease (e.g., cancer relapse or metastasis), alleviating the symptoms or arresting or inhibiting further development of the disease, condition, or disorder. The term “treating” or “alleviating” further includes the administration of compounds or agents to a subject to enhance the efficacy of or restore responsiveness to another therapy. Subjects in need of treatment include those already suffering from the disease or disorder as well as those being at risk of developing the disorder. Treatment may be prophylactic (to prevent or delay the onset of the disease, or to prevent the manifestation of clinical or subclinical symptoms thereof) or therapeutic suppression or alleviation of symptoms after the manifestation of the disease. In the treatment of a disease or disorder, a therapeutic agent may directly decrease the pathology of the disease, or render the disease more susceptible to treatment by other therapeutic agents.

“In combination with”, “combination therapy” and “combination products” refer, in certain embodiments, to the concurrent administration to a subject of a first therapeutic agent (e.g., a known anti-cancer drug) and a second therapeutic agent (e.g., a NAD⁺-upregulating compound described herein). Unless otherwise specified, each component can be administered at the same time or sequentially in any order at different points in time. Thus, each component can be administered separately but sufficiently closely in time so as to provide the desired therapeutic effect. “Concomitant administration” of a known drug for treating cancer with a pharmaceutical composition of the present invention means administration of the drug and the composition which includes a NAD⁺-upregulating compound at such time that both the known drug and the composition of the present invention will have a therapeutic effect. Such concomitant administration may involve concurrent (i.e. at the same time), prior, or subsequent administration of the known anti-cancer drug with respect to the administration of a NAD⁺-upregulating compound of the present invention. A person of ordinary skill in the art would have no difficulty determining the appropriate timing, sequence and dosages of administration for particular drugs and compositions of the present invention.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

As used herein, the term “administration” refers to the act of giving a drug, prodrug, antibody, or other agent, or therapeutic treatment to a physiological system (e.g., a subject or in vivo, in vitro, or ex vivo cells, tissues, and organs). Exemplary routes of administration to the human body can be through the eyes (opthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

As used herein, the term “neoplastic disease” refers to any abnormal growth of cells or tissues being either benign (non-cancerous) or malignant (cancerous).

As used herein, the term “regression” refers to the return of a diseased subject, cell, tissue, or organ to a non-pathological, or less pathological state as compared to basal nonpathogenic exemplary subject, cell, tissue, or organ. For example, regression of a tumor includes a reduction of tumor mass as well as complete disappearance of a tumor or tumors.

Tumor grade is the description of a tumor based on how abnormal the tumor cells and the tumor tissue look under a microscope. It is an indicator of how quickly a tumor is likely to grow and spread. If the cells of the tumor and the organization of the tumor's tissue are close to those of normal cells and tissue, the tumor is called “well-differentiated.” These tumors tend to grow and spread at a slower rate than tumors that are “undifferentiated” or “poorly differentiated,” which have abnormal-looking cells and may lack normal tissue structures. Based on these and other differences in microscopic appearance, doctors assign a numerical “grade” to most cancers. The factors used to determine tumor grade can vary between different types of cancer.

Tumor grade is not the same as the stage of a cancer. Cancer stage refers to the size and/or extent (reach) of the original (primary) tumor and whether or not cancer cells have spread in the body. Cancer stage is based on factors such as the location of the primary tumor, tumor size, regional lymph node involvement (the spread of cancer to nearby lymph nodes), and the number of tumors present.

Grading systems differ depending on the type of cancer. In general, tumors are graded as 1, 2, 3, or 4, depending on the amount of abnormality. In Grade 1 tumors, the tumor cells and the organization of the tumor tissue appear close to normal. These tumors tend to grow and spread slowly. In contrast, the cells and tissue of Grade 3 and Grade 4 tumors do not look like normal cells and tissue. Grade 3 and Grade 4 tumors tend to grow rapidly and spread faster than tumors with a lower grade.

For breast cancer, the Nottingham grading system (also called the Elston-Ellis modification of the Scarff-Bloom-Richardson grading system) is usually used. This system grades breast tumors based on the following features: (1) Tubule formation: how much of the tumor tissue has normal breast (milk) duct structures; (2) Nuclear grade: an evaluation of the size and shape of the nucleus in the tumor cells; and (3) Mitotic rate: how many dividing cells are present, which is a measure of how fast the tumor cells are growing and dividing. Each of the categories gets a score between 1 and 3; a score of “1” means the cells and tumor tissue look the most like normal cells and tissue, and a score of “3” means the cells and tissue look the most abnormal. The scores for the three categories are then added, yielding a total score of 3 to 9. Three grades are possible: (1) Total score=3-5: G1 (Low grade or well differentiated); (2) Total score=6-7: G2 (Intermediate grade or moderately differentiated); and (3) Total score=8-9: G3 (High grade or poorly differentiated).

### III. Resensitizing Refractory Cancer to Anti-Hormone Therapy

Hormone therapy (or anti-hormone therapy) is a form of systemic therapy commonly used for treating ER-positive cancer (e.g., ER-positive breast cancer). It is most often used as an adjuvant therapy to help reduce the risk of the cancer coming back after surgery, but it can be used as neoadjuvant treatment as well. It is also used to treat cancer that has come back after treatment or that has spread. A woman's ovaries are the main source of the hormone estrogen until menopause. After menopause, smaller amounts are still made in the body's fat tissue, where a hormone made by the adrenal gland is converted into estrogen. Estrogen promotes the growth of cancers that are hormone receptor positive. About 2 out of 3 of breast cancers are hormone receptor positive—they contain receptors for the hormones estrogen (ER-positive cancers) and/or progesterone (PR-positive cancers). Most types of hormone therapy for breast cancer either stop estrogen from acting on breast cancer cells or lower estrogen levels. This kind of treatment is helpful for hormone receptor-positive breast cancers, but it does not help patients whose tumors are hormone receptor negative (both ER- and PR-negative).

The studies described herein demonstrate that enhanced NAMPT activation or expression induction, or enhancement of the NAD⁺ synthesis and salvage pathways or NAD⁺ levels, can drastically reduce treatment resistance and recurrence of ER-positive cancer (e.g., breast cancer or ovarian cancer) treated with anti-hormone therapy. They further indicate that NAD⁺ precursor treatment can re-sensitize and sensitize tumor cells to anti-hormone therapy that have become refractory to or were previously not responsive to this therapy, including ER-positive and ER-negative cancer cells, and that the treatment could benefit patients harboring tumor cells that are or have become resistant to anti-hormone treatment. The invention accordingly provides methods for re-sensitizing treatment-resistant cancer cells to an anti-hormone drug or re-sensitizing a subject afflicted with treatment-refractory cancer cells to anti-hormone treatment. In some preferred embodiments, the patients to be treated have already undergone hormone therapy and, in the process, have developed resistance to continuing adjuvant anti-hormone treatment. The compositions of the invention can potentiate sensitivity of cancer cells to further treatment with adjuvant anti-hormone drugs. In some embodiments, a patient may be sequentially or simultaneously treated with a hormone therapy and a NAD⁺-upregulating composition of the invention.

Patients who are undergoing or have undergone hormone therapy via various drugs are suitable for treatment with methods of the invention. These include tamoxifen, aromatase inhibitors, and estrogen receptor downregulators such as Fulvestrant. For example, the patient can be previously or concurrently treated with Tamoxifen along with a therapeutic composition of the invention. Tamoxifen blocks estrogen receptors in breast cancer cells. This stops estrogen from binding to them and telling the cells to grow and divide. While tamoxifen acts like an anti-estrogen in breast cells, it acts like an estrogen in other tissues, like the uterus and the bones. Because it acts like estrogen in some tissues but like an anti-estrogen in others, it is called a selective estrogen receptor modulator or SERM. For women with hormone receptor-positive invasive breast cancer, taking tamoxifen after surgery for 5 years reduces the chances of the cancer coming back by about half, and helps patients live longer. It also lowers the risk of a new breast cancer in the other breast. Some recent studies have shown that taking tamoxifen for 10 years can be even more helpful. For women who have been treated for ductal carcinoma in situ (DCIS) that is hormone receptor-positive, taking tamoxifen for 5 years lowers the chance of the DCIS coming back. It also lowers the chance of getting an invasive breast cancer. Tamoxifen can also stop the growth and even shrink tumors in women with metastatic breast cancer. It can also be used to reduce the risk of developing breast cancer in women at high risk.

Other examples of anti-hormone treatment drugs include Toremifene (Fareston®), Fulvestrant (Faslodex®), Aromatase inhibitors (AIs), Megestrol acetate (Megace®) and Androgens (male hormones). Toremifene is a drug similar to tamoxifen. It is also a SERM and has similar side effects. It is only approved to treat metastatic breast cancer. This drug is not likely to work if tamoxifen has been used and stopped working. Fulvestrant is a drug that first blocks the estrogen receptor and then also eliminates it temporarily. It is not a SERM—it acts like an anti-estrogen throughout the body. Fulvestrant is used to treat advanced (metastatic breast cancer), most often after other hormone drugs (like tamoxifen and often an aromatase inhibitor) have stopped working. It is currently approved by the FDA only for use in post-menopausal women with advanced breast cancer that no longer responds to tamoxifen or toremifene. It is sometimes used “off-label” in pre-menopausal women, often combined with a luteinizing-hormone releasing hormone (LHRH) agonist to turn off the ovaries (see below).

Aromatase inhibitors (AIs) are drugs which can lower estrogen levels in patients. Three drugs that stop estrogen production in post-menopausal women have been approved to treat both early and advanced breast cancer: letrozole (Femara), anastrozole (Arimidex), and exemestane (Aromasin). They work by blocking an enzyme (aromatase) in fat tissue that is responsible for making small amounts of estrogen in post-menopausal women. They cannot stop the ovaries from making estrogen, so they are only effective in women whose ovaries aren't working (like after menopause). These drugs are taken daily as pills. So far, each of these drugs seems to work as well as the others in treating breast cancer. Several studies have compared these drugs to tamoxifen as adjuvant (after surgery) hormone therapy in post-menopausal women. Using these drugs, either alone or after tamoxifen, has been shown to better reduce the risk of the cancer coming back later than using just tamoxifen for 5 years.

Megestrol acetate (Megace®) is a progesterone-like drug that can be used as a hormone treatment of advanced breast cancer, usually for women whose cancers do not respond to the other hormone treatments. Its major side effect is weight gain, and it is sometimes used in higher doses to reverse weight loss in patients with advanced cancer. Androgens (male hormones) may rarely be considered after other hormone treatments for advanced breast cancer have been tried. They are sometimes effective, but they can cause masculine characteristics to develop such as an increase in body hair and a deeper voice.

Patients who have never undergone hormone therapy due to the lack of estrogen receptor alpha at diagnosis are also suitable for treatment with methods of the invention. These include the use of tamoxifen or aromatase inhibitors in conjunction with an agent to up-regulate NAD+ or NAMPT. Cancer cells, including triple negative breast cancer, can express other estrogen receptor such as estrogen receptor beta as a potential target of anti-hormone therapy. The compositions of the invention can sensitize ER alpha-negative (ER negative) breast cancer cells to treatment with adjuvant anti-hormone drugs. This provides a new treatment option for this group of patients who at present can only be subject to toxic and inefficient treatments.

### IV. Enhancing Hormone Therapy Efficacy by Upregulating NAMPT or NAD⁺

The invention provides compositions and therapeutic regimens that are useful in combination with hormone therapy (adjuvant anti-hormone therapy) for treating patients suffering from or at risk of developing cancer. Some compositions of the invention contain a combination of agents for anti-hormone therapy (e.g., tamoxifen) and agents for upregulating NAD⁺ or NAD⁺/NADH redox as described herein. In some aspects, the therapeutic agents described herein are employed to enhance efficacy in anti-hormone treatment of breast cancer and ovarian cancer. In breast cancer, 75% of new cases (173,880/year in the US) will be ER⁺ and treatable with anti-hormone therapy. Of these ER⁺ cases, 40% will not respond to anti-hormone therapy. In ovarian cancer, 86% of new cases (18,309/year in the US) will be ER⁺. As exemplified herein with treatment by NAD⁺ precursors, the inventors demonstrated that the efficacy of anti-hormone therapy can be enhanced by means to activate NAMPT, to induce NAMPT expression, to enhance NAD⁺ synthesis and salvage pathway, or to otherwise upregulate NAD⁺ levels. NAMPT activation or expression induction, enhancement of the NAD⁺ synthesis and salvage pathways, or upregulation of NAD⁺ levels via other means can drastically reduce resistance and recurrence of ER-positive cancer (e.g., breast cancer or ovarian cancer) treated with anti-hormone therapy. The invention accordingly provides therapeutic methods which combine anti-hormone therapy with a regiment that upregulates NAD⁺ level (or NAD⁺/NADH redox balance) or NAMPT activities (enzyme activation or expression induction).

The therapeutic regimen can also be used in the prevention of recurrence and progression of ER-positive cancers and other tumors in patients treated with anti-hormone therapy. For example, modulation of NAD⁺/NADH metabolism through NAD⁺ precursor treatment can be used to prevent ER-positive breast cancer relapse when combined with standard of care to extend the indolence period characterized by absence of clinical disease symptoms, and slow cancer progression, overall extending patient survival. Standard of care therapies whose efficacy will benefit from modulation of NAD⁺ metabolism are anti-hormone therapies described herein, such as anti-estrogens (e.g. tamoxifen), aromatase inhibitors, and estrogen receptor downregulators such as Fulvestrant. These are main therapies that are widely used for patients with ER-positive breast cancers, in the pre- and post-menopausal setting. Anti-hormone therapy is also used to prevent breast cancer in women at high risk.

The therapeutic methods of the invention can also be used in the prevention of recurrence and progression of ER-negative breast cancer. For example, induction of NAD⁺ levels through NAD⁺ precursor treatment can be used to prevent triple-negative breast cancer relapse following surgical removal of the primary tumor and/or radiation or chemotherapy treatment. Furthermore, NAD⁺ precursor treatment can sensitize triple-negative breast cancer to anti-hormone therapy. A combination of NAD⁺ precursors and anti-hormone cotreatment can reduce tumor recurrence and extend patient survival.

In any of these settings, modulation of NAD⁺ metabolism can support prevention of cancer development or cancer growth (e.g., breast cancer), enhance therapeutic efficacy of anti-hormone therapy for patients with cancer, and prevent disease recurrence after anti-hormone therapy, in addition to re-sensitizing tumor cells that are or have become resistant to anti-hormone therapy, and sensitizing triple-negative tumor cell to anti-hormone therapy as described above. This applies to patients with invasive or non-invasive primary tumors, before and after surgical removal of primary tumors, as well as to patients with metastatic disease. Thereby, therapeutic modulation of NAD⁺ metabolism can synergize with standard of care and prolong patient survival. In addition to use in breast cancer patients, anti-estrogens such as tamoxifen or aromatase inhibitors are also useful to treat patients with other solid tumor such as ovarian cancers. Patients with these solid tumors that have been treated with anti-hormone therapy will also benefit from a NAD⁺ upregulating treatment in combination with anti-hormone therapy.

Patients with ER-positive tumors that express high levels as well as those that express low levels of NAMPT would both benefit from combining anti-hormone and NAD⁺ upregulating treatment (e.g., via NAD⁺ precursors or NAMPT expression induction). For patients with low NAMPT expressing tumors, who have the poorer prognosis, such a combined treatment can significantly extend survival. The therapeutic regimen of the invention can drastically reduce resistance to anti-hormone treatment of ER positive cancers (e.g., breast cancers and ovarian cancers) and block recurrence of cancers that were previously treated with anti-hormone therapy. In particular, most breast cancers are ER-positive and thus are often treated with anti-hormone therapy. Enhancement of the efficacy of this therapy and prevention of treatment resistance and disease recurrence through the methods of the invention can significantly enhance survival in breast cancer patients. In addition, based on the inventors' observation that NAD⁺ precursor treatment enhances the anti-proliferative effects of anti-hormone therapy, NAD⁺ upregulation could enable clinical efficiency of lower anti-hormone therapy doses and allow for extended use of anti-hormone therapy beyond the 5-year mark to optimize overall outcome while preserving quality of life.

In addition, patients with triple negative tumors can also benefit from the new combinatory treatment option. As described herein, the therapeutic regimen of the invention can prevent triple negative tumor recurrence and significantly enhance patient survival.

In general, therapeutic methods of the invention utilize agents which can ultimately upregulate NAD⁺ levels to enhance efficacy of cancer treatment. In various embodiments of the invention, an agent capable of upregulating NAD⁺ level or NAD⁺/NADH redox ratio is administered to a cancer patient who has undergone or is undergoing treatment via anti-hormone therapy. In some preferred embodiments, the agents are employed to enhance efficacy of anti-hormone treatment of breast cancer or ovarian cancer. As detailed below, NAD⁺ upregulation can be achieved by, e.g., enhancing NAMPT expression or cellular levels, or by boosting NAD⁺ synthesis or NAD⁺/NADH redox balance. In some of these embodiments, the methods rely on directly upregulating the NAD⁺ level or NAD⁺/NADH redox balance (the ratio of NAD⁺/NADH levels) via the use of NAD⁺ precursors. In some other embodiments, the therapeutic effect is achieved, e.g., by NAMPT activation through induction of NAMPT expression.

Upregulated NAD⁺ levels or NAD⁺/NADH redox balance in tumor cells can be achieved via various means. These include modulation of both NAD⁺/NADH redox pathways and non-redox pathways. These pathways can all be modulated in accordance with methods or protocols well known in the art or described herein. A number of NAD⁺/NADH redox pathways can be modulated to upregulate the NAD⁺/NADH redox balance in the present invention. Once NAD⁺ is synthesized, it is either reduced to NADH and serves as an electron carrier, or it is phosphorylated to NADP⁺ to be further reduced to NADPH. NADH and NADPH are oxidized in catabolic reactions. The NADP⁺/NADPH balance will affect cellular NAD⁺/NADH redox status (see, e.g., Ying, Antioxid. Redox Signal. 10, 179-206, 2008). Therapeutically targetable pathways that modulate the cellular NAD⁺/NADH redox balance, such as catabolic and anabolic pathways include glycolysis pathway, pentose phosphate pathways, and cytosolic NAD⁺ regeneration pathways.

Aerobic glycolysis (Warburg effect) is probably the most general metabolic alteration found in tumor cells. Glycolysis generates ATP, NADH and key metabolic intermediates. NADH from NAD⁺ is generated by GAPDH. The pentose phosphate pathway is important for generation of NADPH (e.g., for fatty acid synthesis and recovery of gluthathione) and key intermediates for nucleotide biosynthesis, including NAD⁺. The pentose phosphate pathway is not an energy pathway, but fed by glycolytic intermediate glucose-6-P. Activation of this pathway regulates the flow of glycolysis, which can be controlled by tumor suppressor p53.

Modulation of pathways of cytosolic NAD⁺ regeneration and NADH cytosolic/mitochondria shuttle is also suitable for the invention. High glycolysis rates decrease levels of NAD⁺. Consequently, NAD⁺ dependent metabolic reactions like glycolysis itself and serine synthesis are dramatically reduced. To recover NAD⁺ in the cytosol, cells use 3 pathways: a) Lactate Dehydrogenase, highly active in tumor cells. b) Glycerol 3-P Shuttle which moves one electron from cytosolic NADH to mitochondrial FADH₂, which feeds mitochondrial complex II. The capacity of the glycerol 3-P shuttle was found reduced in tumor cells. c) Malate-Aspartate Shuttle, an alternative pathway to move one electron from cytosolic NADH to mitochondrial NADH. In mitochondria, NAD⁺ is regenerated from NADH by complex I.

Other NAD⁺/NADH redox pathways suitable for modulation in the practice of the invention include lipid synthesis, citric acid cycle (TCA) pathway, glutaminolysis, beta-oxidation pathway, mitochondrial respiration pathway, and nicotinamide nucleotide transhydrogenase (NNT). Modulation of any of these pathways can all directly or indirectly alter the NAD⁺/NADH redox balance. NADPH is oxidized to NADP⁺ during lipid synthesis (Kaelin et al., Nature 465, 562-4, 2010). The TCA cycle is a central source of metabolic intermediates, and NADH and FADH₂ which feed OXPHOS into complex I and complex II, respectively. For the glutaminolysis pathway, tumors use high levels of glutamine to produce energy through generation of NADH, and to generate key metabolic intermediates. Beta-oxidation pathway generates NADH and FADH₂ which feed OXPHOS after transport of fatty acid into mitochondria through the carnitine shuttle. Regarding the mitochondrial respiration pathway, enhancement of mitochondrial activity results in increased NAD⁺/NADH ratios (Santidrian et al., J. Clin. Invest. 123: 1068-1081, 2013). Measures to enhance mitochondrial activity include approaches to induce or mimic caloric restriction or glucose deprivation. Furthermore, measures to enhance mitochondrial complex I activity, e.g., treatment with selenium or resveratrol, result in increased NAD⁺/NADH ratios, which in turn can enhance the efficacy of anti-hormone therapy. See, e.g., Mehta, mitochondrial biogenesis, and reduces infarct volume after focal cerebral ischemia. BMC Neurosci. 13, 79, 2012; and Desquiret-Dumas et al., J. Biol. Chem. 288, 36662-75, 2013. Finally, nicotinamide nucleotide transhydrogenase (NNT) is a proton pumping enzyme located in the mitochondria that reduces NADP⁺ to NADPH, using NADH as an electron donor and increases NAD⁺ levels in the mitochondria. See, e.g., Gameiro et al., J. Biol. Chem. 288, 12967-77, 2013; and Sites et al., J. Biol. Chem. 288, 12978-12978, 2013; and Olgun, Biogerontology 10, 531-4, 2009.

Other than NAD⁺/NADH redox pathways, enhanced NAD⁺ levels or NAD⁺/NADH redox balance in tumor cells can also be realized by modulating NAD⁺ non-redox pathways in the practice of the invention. These include, e.g., NAD⁺ synthesis or NAD⁺ consumption pathways. Once synthesized, NAD⁺ can be consumed by NAD⁺ dependent enzymes (mainly PARPs, Sirtuins, or CD38). There are multiple opportunities to achieve therapeutic enhancement of tumor cell NAD⁺ metabolism by modulating NAD⁺ synthesis or consumption pathways that will regulate NAD⁺ dependent enzymatic pathways.

Modulation of NAD⁺ synthesis can be carried out by using NAD⁺ precursors. Cellular NAD⁺ levels are controlled by NAD⁺ biosynthesis from precursors, mainly NAM and NIC, but also by nicotinamide riboside (NR) and tryptophan. Other potential precursors include NAD⁺ intermediate metabolites such as, kynurenine, 2-amino-3-carboxymuconic-6-semialdehyde decarboxylase, quinolinic acid, nicotinic acid mononucleotide, nicotinic acid adenine dinucleotide, nicotinamide mononucleotide (Ying, Antioxid. Redox Signal. 10, 179-206, 2008). Regulation of NAD⁺ synthesis can also be achieved by modulating expression of activities of enzymes involved in the synthesis of NAD⁺. Examples of such enzymes include NRK1, NRK2, QPET, NAPRT, NMNAT1, NMNAT2 and NMNAT3. See, e.g., Chiarugi et al., Nat. Rev. Cancer 12, 741-52, 2012.

Modulation of NAD⁺ levels in the practice of the invention can also be achieved by regulating NAD⁺ consumption pathways. In addition to hundreds of metabolic reactions, NAD⁺ is also used by NAD⁺ consuming enzymes, such as PARPs, Sirtuins and CD38. See, e.g., Koch-Nolte et al., FEBS Lett. 585, 1651-6, 2011; Xu et al., Mech. Ageing Dev. 131, 287-98, 2010; and Imai et al., Diabetes. Obes. Metab. 15 Suppl 3, 26-33, 2013; Zhang et al., J. Biol. Chem. 284, 20408-17, 2009; Zhang et al., J. Bioanal. Biomed. 3: 13-25, 2011; Galli et al., Cancer Res. 70, 8-11, 2010; and Kirkland, Curr. Pharm. Des. 15, 3-11, 2009. Modulation of the expression of enzymatic activities of any of these enzymes can also lead to altered NAD⁺/NADH redox balance in tumor cells.

Other than directly modulating NAD⁺ level or NAD⁺/NADH levels, the methods of the invention can also employ compounds or means which can boost NAMPT expression or cellular levels. For example, the methods can use gene therapy to enhance NAMPT levels to prevent tumor relapse after anti-hormone therapy. The gene therapy can utilize, e.g., tumor cell specific delivery of a therapeutic transgene encoding NAMPT for targeted expression of NAMPT. Alternatively, enhanced NAMPT expression can be achieved via stem cell-based gene delivery or tumor marker targeted gene delivery.

In some other embodiments, the agents employed in the therapeutic methods of the invention are compounds which can induce glucose deprivation to enhance NAMPT expression. These include treatments that can decrease glucose levels in blood such as Metformin, treatments that can inhibit the use of glucose by tumor cells such as 2-deoxyglucose, and treatments that can reduce insulin or IGF levels.

### V. Prognosing, Diagnosing and Monitoring Outcome of Hormone Therapy

As demonstrated by the inventors, high NAMPT levels correlate with good prognosis and outcome in tamoxifen treated ER-positive breast cancer patients. Similarly, high NAD⁺ level or high ratio of NAD⁺/NADH levels also correlate with a low risk of cancer relapse after anti-hormone therapy. Thus, NAMPT expression levels and/or NAD⁺ levels in tumors can be important indicators to identify patients who, when treated with anti-hormone therapy, have a high risk of progressing under treatment or relapsing after treatment stops. These measures can identify patients who would need and benefit most from additional treatments to enhance survival. For example, identification of patients with low NAMPT level and/or low NAD⁺ level in the tumor, who are likely to experience cancer relapse after anti-hormone therapy, will facilitate the adoption of early alternative/additional strategies to treat patients with ER-positive cancers and improve overall outcome.

The invention accordingly provides methods for prognosis, diagnosis and monitoring of hormone therapy outcome or treatment effect (e.g., cancer recurrence and metastasis) in patients who have undergone, are undergoing or will undergo anti-hormone therapy for cancer. In general, diagnosis is the determination of the present condition of a patient (e.g., presence or absence of relapse) and prognosis is developing future course of the patient (e.g., risk of developing relapse in the future or likelihood of improvement in response to treatment). In some embodiments, cancer patients (e.g., subjects afflicted with breast cancer or ovarian cancer) can be examined with such methods of the invention to diagnose or prognose likely effects of anti-hormone treatment. In some preferred embodiments, the methods are directed to diagnosis or prognosis of anti-hormone therapy of breast cancer, especially ER-positive breast cancer. The treatment effects that can be monitored with methods of the invention include, e.g., risk of relapse after the therapy, distant metastasis and survival.

The diagnosis or prognosis methods of the invention typically entail measuring NAMPT expression or cellular level, NAD⁺ level, or ratio of NAD⁺/NADH levels, in the tumor cells present in or obtained from the subject. The measurement is preferably performed prior to commencement of the anti-hormone treatment. Additional measurements can also be taken during the treatment and subsequent to the treatment. By comparing the measured NAMPT expression level (or NAD⁺ level, or ratio of NAD⁺/NADH levels) in the tumor to a standard or reference level, the prognosis methods allow identification of patients with breast cancer (or ovarian cancer) who are at increased risk of relapse after anti-hormone therapy. This can facilitate the adoption of early alternative or additional means to treat patients with cancers and improve overall outcome.

Measurement of NAMPT expression level (or NAD level, or ratio of NAD⁺/NADH levels) in the tumor can be performed via standard techniques routinely practiced in the art or specifically exemplified herein. Expression level of NAMPT can be measured at the protein or nucleic acid level. The measured level can be absolute in terms of a concentration of an expression product, or relative in terms of a relative concentration of an expression product of interest to another expression product in the sample. For example, relative expression levels of genes can be expressed with respect to the expression level of a house-keeping gene in the sample. Expression levels can also be expressed in arbitrary units, for example, related to signal intensity.

Using NAMPT expression level as an example, the individual expression levels, whether absolute or relative, can be converted into values or other designations providing an indication of presence or risk of relapse or metastasis by comparison with one or more reference points. The reference points can include a measure of an average expression level of NAMPT in subjects having had anti-hormone therapy without relapse or metastasis, and/or an average value of expression levels in subjects having had anti-hormone therapy with relapse or metastasis. The reference points can also include a scale of values found in cancer patients who have undergone anti-hormone therapy including patients having and not having cancer recurrence. Such reference points can be expressed in terms of absolute or relative concentrations as for measured values in a sample.

For comparison between a measured NAMPT expression level and reference level(s), the measured level sometimes needs to be normalized for comparison with the reference level(s) or vice versa. The normalization serves to eliminate or at least minimize changes in expression level unrelated to cancer relapse or metastasis (e.g., from differences in overall health of the patient or sample preparation). Normalization can be performed by determining what factor is needed to equalize a profile of expression levels measured in a sample with expression levels in a set of reference samples from which the reference levels were determined. Commercial software is available for performing such normalizations between different sets of expression levels.

Comparison of the measured NAMPT expression level with one or more of the above reference points provides a value (i.e., numerical) or other designation (e.g., symbol or word(s)) of likelihood or susceptibility to cancer relapse. In some methods, a binary system is used; that is a measured expression level of a gene is assigned a value or other designation indicating presence or susceptibility to cancer relapse or lack thereof without regard to degree. For example, the expression level can be assigned a value of +1 to indicate presence or susceptibility to cancer relapse and −1 to indicate absence or lack of susceptibility to cancer relapse. Such assignment can be based on whether the measured expression level is closer to an average level in breast cancer patients having or not having cancer relapse. In other methods, a ternary system is used in which an expression level is assigned a value or other designation indicating presence or susceptibility to cancer relapse or lack thereof or that the expression level is uninformative. Such assignment can be based on whether the expression level is closer to the average level in breast cancer patient undergoing cancer relapse, closer to an average level in breast cancer patients lacking cancer relapse or intermediate between such levels. For example, the expression level can be assigned a value of +1, −1 or 0 depending on whether it is closer to the average level in patients undergoing cancer relapse, is closer to the average level in patients not undergoing cancer relapse or is intermediate. In other methods, a particular expression level is assigned a value on a scale, where the upper level is a measure of the highest expression level found in breast cancer patients and the lowest level of the scale is a measure of the lowest expression level found in breast cancer patients at a defined time point at which patients may be susceptible to cancer relapse (e.g., one year post surgery). Preferably, such a scale is a normalized scale (e.g., from 0-1) such that the same scale can be used for different genes. Optionally, the value of a measured expression level on such a scale is indicated as being positive or negative depending on whether the upper level of the scale associates with presence or susceptibility to cancer relapse or lack thereof. It does not matter whether a positive or negative sign is used for cancer relapse or lack thereof, as long as the usage is consistent for different genes.

In some embodiments, both NAMPT expression level and ratio of NAD′/NADH levels can be measured in the tumor in order to provide a prognosis of effects of anti-hormone therapy in the patients. In these methods, the values or designations obtained for NAMPT expression level and ratio of NAD⁺/NADH levels can be combined to provide an aggregate value. If each level is assigned a score of +1 if its expression level indicates presence or susceptibility to cancer relapse, and −1 if its expression level indicates absence or lack of susceptibility to cancer relapse and optionally zero if uninformative, the different values can be combined by addition. The same approach can be used if each level is assigned a value on the same normalized scale and assigned as being positive or negative, depending whether the upper point of the scale is associate with presence or susceptibility to cancer relapse or lack thereof. Other methods of combining values for individual biomarkers of disease into a composite value that can be used as a single marker are described in US20040126767 and WO/2004/059293.

The above described methods can provide a value or other designation for a patient which indicates whether the aggregate measured levels in a patient is more likely to have or to develop cancer relapse or metastasis after anti-hormone therapy. Such a value provides an indication that the patient either has or is at enhanced risk of relapse/metastasis, or conversely does not have or is at reduced risk of relapse/metastasis. Risk is a relative term in which risk of one patient is compared with risk of other patients, either qualitatively or quantitatively. For example, the value of one patient can be compared with a scale of values for a population of treated cancer patients having relapse to determine whether the patient's risk relative to that of other patients.

### VI. Pharmaceutical Compositions and Kits

The agents which upregulate NAMPT expression, NAD⁺ level or NAD⁺/NADH redox balance (e.g., a NAD⁺ precursor) and the other therapeutic agents disclosed herein can be administered directly to subjects in need of treatment. However, these therapeutic compounds are preferable administered to the subjects in pharmaceutical compositions which comprise the agents and/or other active agents along with a pharmaceutically acceptable carrier, diluent or excipient in unit dosage form. Accordingly, the invention provides pharmaceutical compositions comprising one or more of the agents disclosed herein. The invention also provides a use of these agents in the preparation of pharmaceutical compositions or medicaments for enhancing hormone therapy efficacy, for re-sensitizing treatment resistant cancer, or for other therapeutic applications described herein. The pharmaceutical compositions of the invention can be used for either therapeutic or prophylactic applications described herein.

Typically, the pharmaceutical compositions contain as active ingredients compounds that specifically upregulate NAMPT expression, NAD⁺ level or NAD⁺/NADH redox balance. Some compositions include a combination of multiple (e.g., two or more) compounds that upregulate NAMPT expression, NAD level or NAD⁺/NADH redox balance. As described herein, the compositions can additionally contain other therapeutic agents that are suitable for treating or preventing cancer relapse or progression. The active ingredients are typically formulated with one or more pharmaceutically acceptable carrier. Pharmaceutically carriers enhance or stabilize the composition, or to facilitate preparation of the composition. They should also be both pharmaceutically and physiologically acceptable in the sense of being compatible with the other ingredients and not injurious to the subject. Pharmaceutically acceptable carriers include solvents, dispersion media, coatings, antibacterial and antifungal agents, isotonic and absorption delaying agents, and the like that are physiologically compatible. The pharmaceutically acceptable carrier employed should be suitable for various routes of administration described herein. For example, the compound that upregulates NAMPT expression (or NAD⁺ level or NAD⁺/NADH redox balance) can be complexed with carrier proteins such as ovalbumin or serum albumin prior to their administration in order to enhance stability or pharmacological properties. Additional guidance for selecting appropriate pharmaceutically acceptable carriers is provided in the art, e.g., Remington: The Science and Practice of Pharmacy, Mack Publishing Co., 20^{th} ed., 2000.

Pharmaceutical compositions of the invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for molecules of the invention include ethylene-vinyl acetate copolymer particles, osmotic pumps, implantable infusion systems, and liposomes. Formulations for inhalation may contain excipients, for example, lactose, or may be aqueous solutions containing, e.g., polyoxyethylene-9-lauryl ether, glycocholate and deoxycholate, or may be oily solutions for administration in the form of nasal drops, or as a gel.

The pharmaceutical compositions can be prepared in various forms, such as granules, tablets, pills, suppositories, capsules, suspensions, salves, lotions and the like. The concentration of therapeutically active compound in the formulation may vary from about 0.1-100% by weight. Therapeutic formulations are prepared by any methods well known in the art of pharmacy. The therapeutic formulations can be delivered by any effective means which could be used for treatment. See, e.g., Goodman & Gilman's The Pharmacological Bases of Therapeutics, Hardman et al., eds., McGraw-Hill Professional (10^{th} ed., 2001); Remington: The Science and Practice of Pharmacy, Gennaro (ed.), Lippincott Williams & Wilkins (20^{th} ed., 2003); and Pharmaceutical Dosage Forms and Drug Delivery Systems, Ansel et al. (eds.), Lippincott Williams & Wilkins (7^{th} ed., 1999).

The agents that upregulate NAMPT expression (or NAD⁺ level or NAD⁺/NADH redox balance) for use in the methods of the invention should be administered to a subject in an amount that is sufficient to achieve the desired therapeutic effect (e.g., eliminating or ameliorating cancer relapse or metastasis) in a subject in need thereof. Typically, a therapeutically effective dose or efficacious dose of the agent is employed in the pharmaceutical compositions of the invention. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular compositions of the present invention employed, the route of administration, the time of administration, and the rate of excretion of the particular compound being employed. It also depends on the duration of the treatment, other drugs, compounds and/or materials used in combination with the particular compositions employed, the age, gender, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. Typically, a pharmaceutically effective dosage would be between about 0.001 and 100 mg/kg body weight of the subject to be treated.

The compounds that upregulate NAMPT expression (or NAD⁺ level or NAD⁺/NADH redox balance) and other therapeutic regimens described herein are usually administered to the subjects on multiple occasions. Intervals between single dosages can be daily, weekly, monthly or yearly. Intervals can also be irregular as indicated by measuring blood levels of the compounds and the other therapeutic agents used in the subject. In some methods, dosage is adjusted to achieve a plasma compound concentration of 1-1000 μg/ml, and in some methods 25-300 μg/ml or 10-100 μg/ml. Alternatively, the therapeutic agents can be administered as a sustained release formulation, in which case less frequent administration is required. Dosage and frequency vary depending on the half-life of the compound and the other drugs in the subject. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the subject can be administered a prophylactic regime.

The invention also provides kits for carrying out the therapeutic applications disclosed herein. For example, the invention provides therapeutic kits for re-sensitizing resistant cancer cells or for treatment of cancer relapse or metastasis in subjects afflicted with ER-positive cancer or ER-negative cancer. The therapeutic kits of the invention typically comprise as active agent one or more of the described compounds that upregulate NAMPT level or NAD⁺/NADH redox balance. The kits can optionally contain suitable pharmaceutically acceptable carriers or excipients for administering the active agents. The pharmaceutically acceptable carrier or excipient suitable for the kits can be coatings, isotonic and absorption delaying agents, binders, adhesives, lubricants, disintergrants, coloring agents, flavoring agents, sweetening agents, absorbants, detergents, and emulsifying agents. Other reagents that can be included in the kits include antioxidants, vitamins, minerals, proteins, fats, and carbohydrates.

The therapeutic kits can further include packaging material for packaging the reagents and a notification in or on the packaging material. The kits can additionally include appropriate instructions for use and labels indicating the intended use of the contents of the kit. The instructions can be present on any written material or recorded material supplied on or with the kit or which otherwise accompanies the kit.

The therapeutic kits of the invention can be used alone in some the therapeutic applications described herein (e.g., enhancing hormone therapy efficacy). They can also be used in conjunction with other known therapeutic regiments. For example, subjects afflicted with an ER-positive or ER-negative cancer can use the therapeutic kit along with a known drug for hormone therapy (e.g., Tamoxifen). The therapeutic composition of the invention and other known treatment regimens can be administered to the subjects sequentially or simultaneously as described herein. These therapeutic applications of the invention can all be indicated on the instructions of the kits.

### EXAMPLES

The following examples are provided to further illustrate the invention but not to limit its scope. Other variants of the invention will be readily apparent to one of ordinary skill in the art and are encompassed by the appended claims.

### Example 1 Metabolic Pathways Affecting Cancer Therapy Efficacy and Resistance

To better understand mechanisms that drive tumor development and cancer progression, we analyzed cellular energy metabolism in breast cancer cells. We particularly focused on NAD⁺ synthesis and salvage pathway due to its possible roles in tumor progression and therapy resistance. We identified specific metabolic pathways which influence therapeutic efficacy and development of resistance to major types of breast cancer treatment.

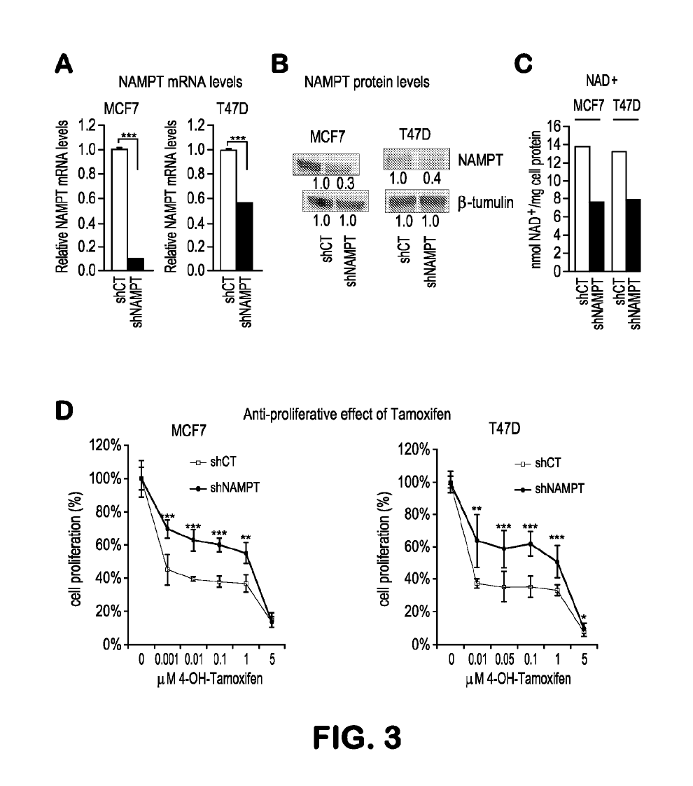

To directly analyze effects of NAMPT expression on the responsiveness of ER-positive breast cancer cells to anti-hormone therapy, we investigated if and how modulation of NAMPT expression affects tumor cell responsiveness to Tamoxifen, a clinically widely used ER antagonist that acts through its active metabolite 4-hydroxytamoxifen. To do this, we first measured NAMPT expression in MCF7 and T47D cells, both luminal A, ER-positive human breast cancer cell lines that require estrogen for proliferation. We found NAMPT was expressed in these cells, and we generated a test model by experimentally reducing NAMPT expression using a shRNA approach. Targeting NAMPT in MCF7 and t47D cells by stable transduction with shRNA (shNAMPT) decreased NAMPT mRNA levels by 83% and 44% respectively (FIG. 3A) and NAMPT protein levels by 70% and 60% respectively (FIG. 3B). Stable transduction with shNAMPT reduced cellular NAD⁺ in both MCF7 and T47D cell lines (FIG. 3C).

Importantly, our results revealed what is opposite to what would been expected based on published studies. Specifically, instead of enhancing tamoxifen efficacy, it was found that reduction of NAMPT expression in ER-positive breast cancer cells dramatically reduced the anti-proliferative efficacy of 4-hydroxytamoxifen in both MCF7 and T47D cells (FIG. 3D). This result is completely unexpected.

### Example 2 Enhancing Anti-Hormone Therapy by Upregulating NAMPT Pathway

The results described in Example 1 are entirely unexpected because they are contrary to what is suggested in the literature (e.g., Hsu et al., Autophagy 5, 1229-1231, 2009) and our own previous studies showing that nicotinamide induces autophagy (Santidrian et al., J. Clin. Invest. 123: 1068-1081, 2013), a mechanism that is thought to inhibit the effects of stress inducing anti-cancer treatments, including anti-hormone therapy. The results described in Example 1 further suggest that activation of the NAMPT pathway might enhance anti-hormone therapy in ER-positive breast cancer cells.

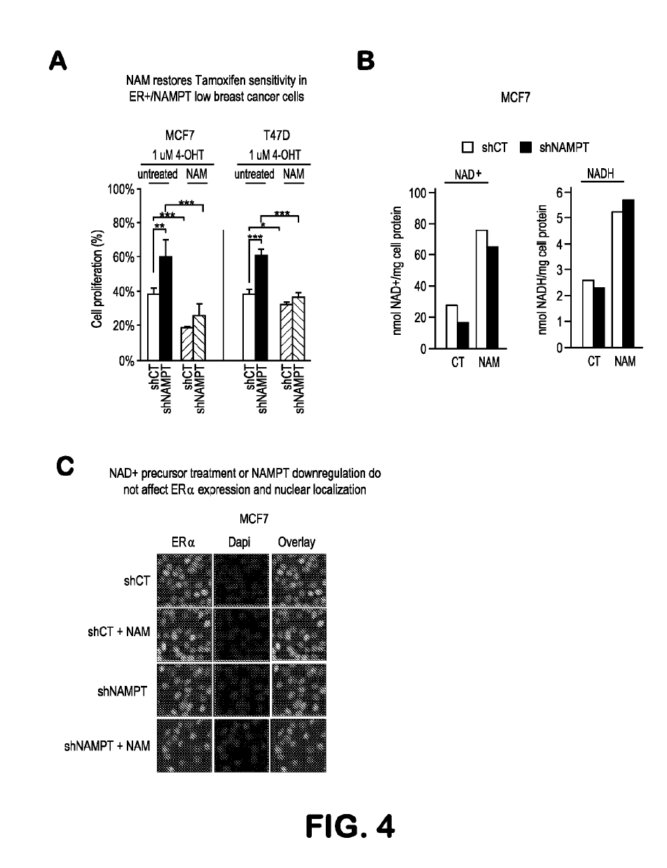

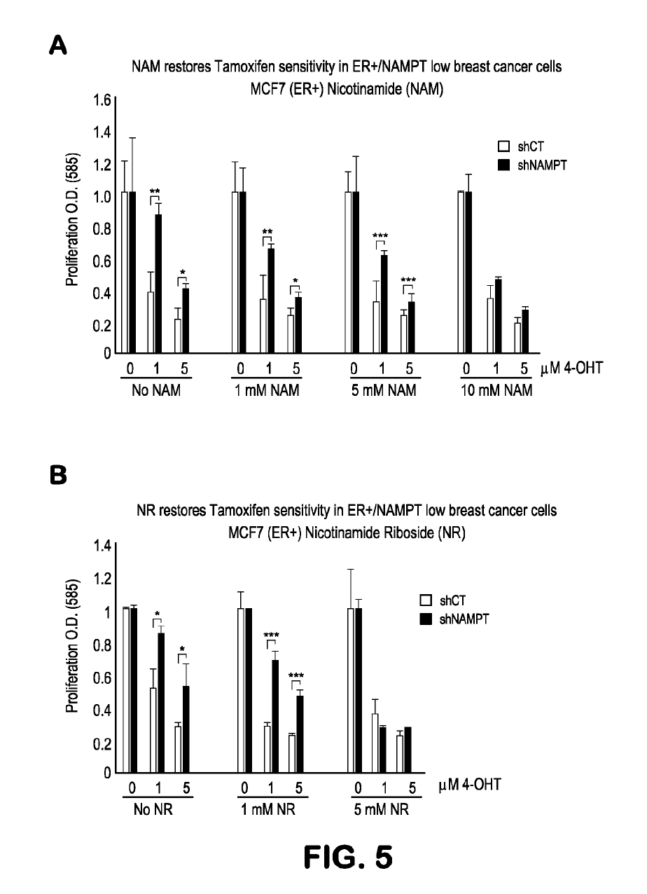

To further analyze this clinically highly relevant finding, which indicates a potential new therapeutic approach, we combined tamoxifen treatment of estrogen positive MCF7 and T47D breast cancer cells with NAM (vitamin B3 and NAD⁺ precursor) treatment. As shown in FIG. 4 A. NAM treatment enhanced the anti-proliferative efficacy of 4-hydroxytamoxifen. Importantly, this was found in control cells expressing endogenous levels of NAMPT, as well as in cells having low levels of NAMPT after experimental reduction of the expression of this gene. Next, we analyzed whether NAM treatment increases NAD⁺ levels in MCF7 cells expressing basal or low NAMPT levels. Interestingly, we found that NAM induced significantly NAD⁺ levels even in the presence of low expression levels of NAMPT (FIG. 4B). NAD⁺ precursor treatment or NAMPT downregulation did not affect ERα expression and nuclear localization in MCF7 cells when the cells were cultured in EMEM medium supplemented with 10% FBS, suggesting that NAD⁺ metabolism might regulate ERα activity rather that expression or localization (FIG. 4C). Moreover, as shown in FIG. 5, nicotinamide riboside (NR), another vitamin B3 and NAD⁺ precursor presented a more potent activity than NAM in restoring Tamoxifen sensitivity in ER+/NAMPT low breast cancer cells. These data indicate that even in the presence of low levels of NAMPT expression, treatment with NAD⁺ precursor significantly enhances the therapeutic efficacy of anti-hormone therapy in ER-positive breast cancer cells. These findings suggest that NAD⁺ precursor treatment sensitizes ER-positive breast cancer cells to tamoxifen treatment, even if NAMPT expression is low; and that NAMPT levels in breast cancer cells can regulate tamoxifen responsiveness of ER-positive breast cancer cells through modulation of NAD⁺ levels.

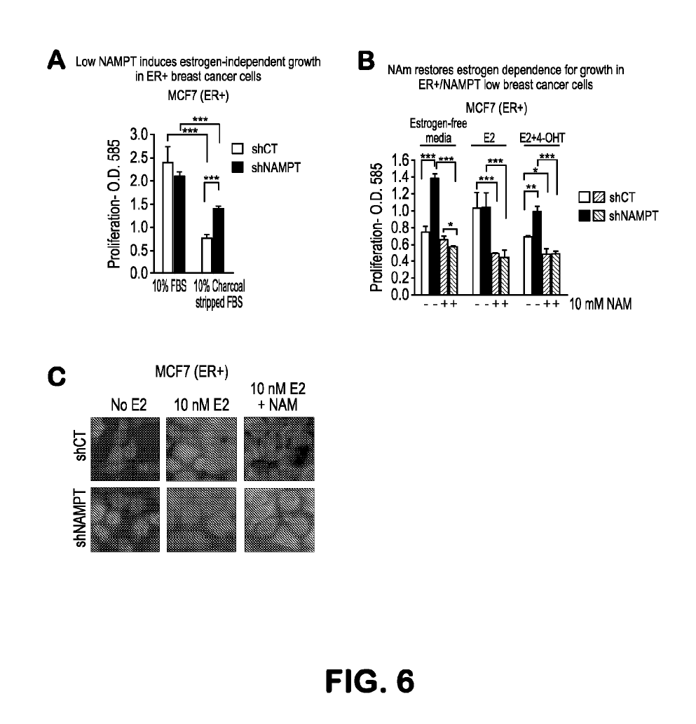

To further analyze the regulatory role of NAMPT and the NAD⁺ salvage pathway in estrogen-dependent growth and tamoxifen treatment, we first analyzed the capacity of MCF7 variant cells to grow in estrogen-free media. We found that the growth of control cells (MCF7shCT) cells was dramatically reduced when cultured in EMEM phenol-red free media (to avoid unspecific estrogen mimetic effects of phenol red) and 10% charcoal stripped serum to eliminate estrogen from the serum. Importantly, low NAMPT expressing MCF7 cells (shNAMPT) had the capacity to proliferate even in the absence of estrogens (FIG. 6A). Interestingly, 17-β-estradiol (E2) induced proliferation in MCF7 shCT cells but not in MCF7 shNAMPT cells, and 4-hydroxytamoxifen treatment was able to reduce proliferation in MCF7 shCT cells but not in shNAMPT (FIG. 6B). Importantly, NAM treatment dramatically inhibited estrogen-induced proliferation in MCF7 shCT cells, estrogen-independent growth of MCF7 shNAMPT cells. Furthermore, NAM treatment sensitized shCT and re-sensitized shNAMPT cells to tamoxifen's antiproliferative effects (FIG. 6B). In FIG. 4C, we analyzed the ERα localization in MCF7 cells when the cells were cultured in EMEM medium supplemented with 10% FBS, serum that contain hormones and growth factors that modulate ERα localization independently of the presence of estrogens (Muriel Le Romancer et al., Endocrine Reviews, October 2011, 32(5):597-622). In order to deeply analyze the role of NAMPT and NAM treatment on modulating ligand-dependent ERα nuclear localization, we starved the cells from any growth factor and hormones including estrogens for 72 hours by growing them in phenol red-free EMEM/10% charcoal-stripped FBS. Then, breast cancer cells were cultured for 24 hours with or without 10 nM E2, or with 10 nM E2 plus 10 mM NAM. Fluorescence imaging of the cells revealed a significant difference in the subcellular localization of ERα in control (shCT) vs. NAMPT knock-down (shNAMPT) cells (FIG. 6C). In the absence of estrogens, ERα was distributed in the cytoplasm of control cells while in shNAMPT expressing cells ERα was concentrated in the nuclei. ERα was localized in the nuclei after stimulation with 10 nM E2. Importantly, 24 h of 10 mM NAM treatment inhibited ERα nuclear localization in control and shNAMPT expressing cells. To completely understand the role of NAMPT and NAD⁺ metabolism in modulating estrogen-independent growth in ER positive cells, we implanted MCF7 shCT and shNAMPT cells into the 4^{th} mammary fat pad in mice, left untreated with 17-β-estradiol pellet to eliminate estrogen-induced tumor growth.

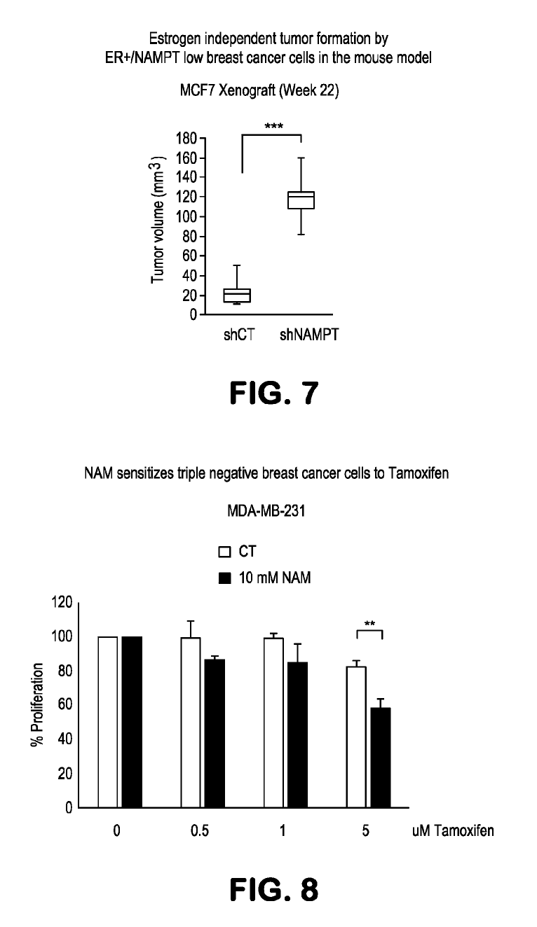

Low NAMPT expression induced tumor growth even without the presence of exogenous implanted estrogens (FIG. 7). MCF7 shCT produced nearly undetectable tumors in these mice that were not treated with 17-β-estradiol. Together, these data indicate that low NAMPT expressing ER-positive tumors became insensitive to estrogens and to anti-hormone therapy. This finding implies that NAD⁺ precursor treatment could drastically reduce resistance and recurrence of ER-positive breast cancers treated with anti-hormone therapy, and thereby significantly enhance survival in breast cancer patients.

To further analyze the potential of this new therapeutic approach, we also treated the triple negative breast cancer cell line MDA-MB-231, known to lack ER alpha receptor expression and to be resistant to anti-hormone therapy, with NAM (vitamin B3 and NAD⁺ precursor) in combination with 4-hydroxytamoxifen. As shown in FIG. 8, NAM treatment sensitized MDA-MB-231 to tamoxifen-induced antiproliferative effects. These data indicate that even in ER negative breast cancer cells, NAD⁺ precursor treatment can significantly induce anti-hormone therapy efficacy, even if the ER alpha is not present. These results demonstrate feasibility of a new treatment option for triple negative breast cancer.

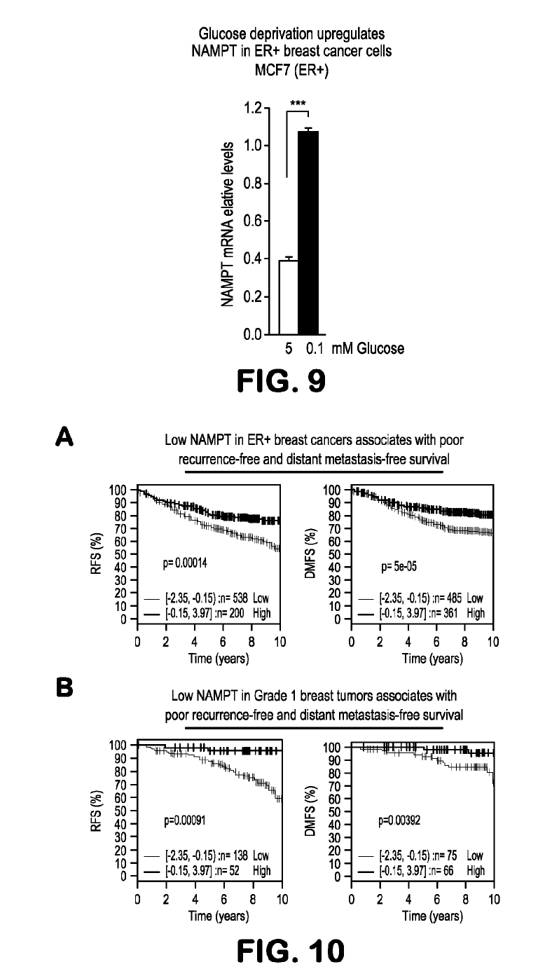

The studies described herein demonstrate that treatment with NAD⁺ precursor nicotinamide significantly and drastically enhances the efficacy of anti-hormone therapy. The findings further indicate that NAMPT plays an important role in the responsiveness of cancer cells to therapy. It has been reported that NAMPT expression is regulated by energy metabolism in the liver, adipose tissue and muscle by circadian rhythm, nutrient intake and exercise. In this context and in light of the importance of NAMPT expression in breast cancer responsiveness to anti-hormone therapy described above, we performed an additional, mechanistically and clinically highly relevant study. We found that glucose deprivation in MCF7 cells, known to induce accumulation of NAD⁺ and to decrease NADH levels, induces NAMPT expression in the tumor cells (FIG. 9). These data demonstrate that energy metabolism regulates NAMPT expression in breast cancer cells. They further indicate that through NAMPT and NAD⁺ related mechanisms, therapeutic enhancement of NAD⁺ metabolism can modulate long-term patient outcome by reducing the rate of disease recurrence after apparently successful anti-hormone treatment. Specifically, the data suggest that NAMPT activation, expression induction or enhancement of the NAD⁺ synthesis and salvage pathways could drastically reduce resistance of ER-positive breast cancers to anti-hormone therapy, and thus inhibit recurrence of breast cancers treated with this major standard-of-care approach. Thereby, enhancement of NAD⁺ synthesis and salvage pathway activity, or modulation of NAMPT activity and expression induction could significantly enhance survival in breast cancer patients.

### Example 3 Prognosing or Diagnosing Efficacy of Anti-Hormone Therapy

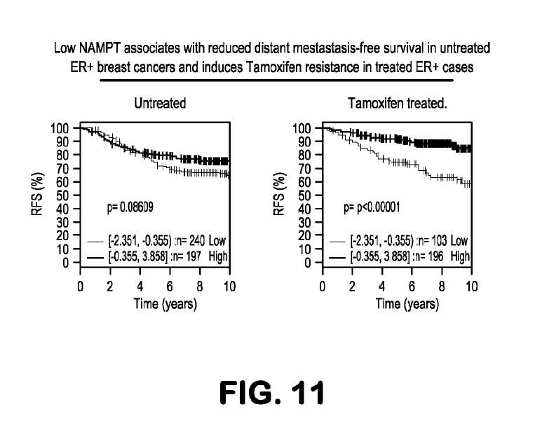

The unexpected results observed by the inventors further suggest that NAMPT expression in breast cancers could be used as a biomarker to monitor the efficacy of anti-hormone therapy, and to determine the probability of tumor recurrence after anti-hormone treatment. To examine clinical evidence for this possibility, we analyzed whether NAMPT expression correlates with anti-hormone therapy outcomes. We used published clinical databases to investigate the relationship between NAMPT expression and prognosis for patients with ER-positive breast cancer (FIGS. 10 and 11). Results from 1881 breast cancer patients (Ringnér et al., PLoS One 6, e17911, 2011) showed that ER-positive breast cancers have significantly lower NAMPT expression levels than ER-negative breast cancers (FIG. 2). Interestingly, our analysis further revealed that ER-positive breast cancers contain a subgroup in which NAMPT expression is high (see box plot distribution in FIG. 2). Within this group of ER-positive breast cancers, relatively high NAMPT expression correlates with good prognosis (FIG. 10A). Furthermore, through careful analysis of individual breast cancer subtypes, we found high NAMPT expression levels are associated with good prognosis in low grade (Grade 1) tumors, independently of receptor status (FIG. 10B). This result is in line with the findings on the ER-positive subgroup, as low grade tumors in general are associated with a better prognosis.

Importantly, by analyzing how NAMPT expression affects the outcome of treatment with tamoxifen, we found that high NAMPT expression in ER-positive breast cancers is associated with a strong increase in relapse-free and distant metastasis-free survival after tamoxifen treatment (FIG. 11). NAMPT expression does not correlate with prognosis in untreated ER-positive breast cancer patients.

These clinical results consolidate our findings that high NAMPT expression correlates with a drastic decrease in tumor recurrence. Importantly, the clinical results further indicate that NAD⁺ precursor treatment could enhance the efficacy of anti-hormone therapy in patients with ER-positive breast cancer and thereby very much improve treatment outcomes and patient survival.

### Example 4 Modulating NAMPT Level to Reduce Breast Cancer Recurrence

Based on our analysis of the gene expression array data in combination with breast cancer subtypes and patient outcomes, we found that high expression levels of NAMPT correlate with a strikingly good prognosis in patients with ER-positive breast cancer after tamoxifen treatment. This suggests that NAMPT expression levels in tumors from patients who will be treated with anti-hormone treatment could be an important measure to identify a group of patients who have a higher risk of relapsing after the treatment stops. The findings also lend support to establishing new non-toxic therapeutic approaches, aimed at enhancing NAD⁺ salvage pathway activity in patients with low NAMPT expression, by introducing changes in nutrition (macronutrients and micronutrients such as vitamin B3 (a NAMPT substrate)) and life style. Instead of inhibiting NAMPT as suggested in the art, the new approaches will rely on NAMPT activation via expression induction or enhancement of the NAD⁺ salvage pathway to achieve the goal of reducing resistance and recurrence of ER-positive breast cancer treated with anti-hormone therapy.

To further confirm feasibility of this approach, studies can be performed in a clinically relevant setting to establish NAMPT levels as a key feature regulated by nutrient intake that could determine ER-positive breast cancer outcome after tamoxifen treatment. The studies will analyze the role of nutrients in regulating NAMPT expression and modulating ER-positive breast cancer responsiveness to anti-hormone therapy.

Specifically, in vitro (cell culture) and in vivo (xenograft models) approaches can be employed to investigate how two macronutrients (glucose and glutamine) and one micronutrient (vitamin B3) can modulate the expression of NAMPT and impact NAD⁺ levels to modulate breast cancer outcome in ER-positive tumor cells treated with anti-hormone therapies. Short-term experiments will mimic the clinical scenario during the time of the treatment. Long-term experiments will mimic the scenario of breast cancer recurrence after treatment stops. Also, the specific role of NAMPT in modulating anti-hormone therapy response and tumor recurrence after treatment stops will be explored by lowering NAMPT expression in vitro and in vivo assays. Moreover, the studies will include investigation on how cellular NAD⁺ metabolism status determines the accumulation of further DNA alterations that could be linked to tumor recurrence. These studies will use standard experimental procedures (e.g., for measuring NAMPT level in cells) and materials as described herein or well known in the art.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

What is claimed is:

1. A method for re-sensitizing or sensitizing a population of treatment resistant cancer cells to an anti-hormone therapy, comprising contacting the treatment resistant cancer cells with a compound that upregulates NAD⁺ or NAD⁺/NADH redox balance in the cells, thereby re-sensitizing or sensitizing the cancer cells.

2. The method of claim 1, wherein the cancer cells are estrogen receptor (ER) positive cells.

3. The method of claim 2, wherein the ER-positive cells are cells of breast cancer or ovarian cancer.

4. The method of claim 1, wherein the cancer cells are estrogen receptor (ER) negative cells.

5. The method of claim 4, wherein the ER-negative cells are cells of breast cancer or ovarian cancer

6. The method of claim 1, wherein the treatment resistant cancer cells are present in a patient.

7. The method of claim 6, wherein the patient has undergone treatment with an anti-hormone therapy.

8. The method of claim 7, wherein the anti-hormone therapy is treatment with Tamoxifen or another compound capable of reducing estrogen levels systemically.

9. The method of claim 1, wherein NAD⁺ or NAD⁺/NADH redox balance is upregulated via enhanced NAD⁺ salvage pathway synthesis, enhanced NAD⁺ de novo synthesis, enhanced NAMPT activation, or enhanced NAMPT cellular level.

10. The method of claim 9, wherein the enhanced NAD⁺ salvage pathway synthesis is via administration of a NAD⁺ precursor.

11. The method of claim 10, wherein the NAD precursor is nicotinamide (NAM), nicotinic acid (Na), or nicotinamide riboside (NR).

12. The method of claim 9, wherein NAD⁺ or NAD⁺/NADH redox balance is upregulated by introducing into the cancer cells an agent that upregulates NAMPT cellular level.

13. The method of claim 12, wherein the agent is a polynucleotide or expression vector encoding NAMPT.

14. The method of claim 13, wherein the polynucleotide is administered to the patient via tumor marker targeted gene delivery.

15. The method of claim 13, wherein the polynucleotide is administered to the patient via stem cell-based gene delivery.

16. The method of claim 12, wherein upregulated NAMPT cellular level is achieved by inducing glucose deprivation in blood or inhibiting consumption of glucose by cancer cells.

17. A method for enhancing anti-hormone therapy efficacy or preventing cancer relapse or progression in a cancer patient, comprising administering to a patient undergoing, having been treated, or never treated with anti-hormone therapy an agent which upregulates NAD⁺ or NAD⁺/NADH redox balance, thereby enhancing anti-hormone therapy efficacy or preventing cancer relapse or progression in the patient.

18-32. (canceled)

33. A method for treating a cancer in a patient, comprising (1) treating the patient with an anti-hormone therapy, and (2) administering to the subject a compound which upregulates NAD⁺ or NAD⁺/NADH redox balance.

34-52. (canceled)

53. A method for prognosing or diagnosing cancer relapse or distant metastasis after anti-hormone therapy in a cancer patient, comprising (a) determining NAMPT level, NAD⁺ level, ratio of NAD⁺/NADH levels in the cancer of the patient, or the level or activity of an enzyme involved in NAD⁺ consumption, and (b) correlating the determined NAMPT level, NAD⁺ level, ratio of NAD⁺/NADH levels, or the level or activity of the enzyme involved in NAD⁺ consumption, with an increased risk of cancer relapse or distant metastasis, or lack thereof, in the patient.

54. The method of claim 53, wherein the enzyme involved in NAD⁺ consumption is PARP, Sirtuins or CD38.

55. A method for prognosing or diagnosing effect of anti-hormone therapy in a cancer patient, comprising (a) determining NAMPT level, NAD⁺ level, ratio of NAD⁺/NADH levels, or the level or activity of an enzyme involved in NAD⁺ consumption, in the cancer of the patient, and (b) prognosing or diagnosing from the determined NAMPT level, ratio of NAD⁺/NADH levels, or the level or activity of the enzyme involved in NAD⁺ consumption, a post-treatment effect of anti-hormone therapy in the patient.

56-62. (canceled)
